(19) United States
(12) Patent Application Publication
(10) Pub. No.: US 2015/0330989 A1
(43) Pub. Date: Nov. 19, 2015

(54) METHOD AND SYSTEM FOR DIAGNOSING AND TREATING PREECLAMPSIA

(71) Applicant: THE BRIGHAM AND WOMEN'S HOSPITAL, INC., Boston, MA (US)

(72) Inventors: Richard M. Burwick, Brookline, MA (US); Bruce Feinberg, Boston, MA (US); Raina N. Fichorova, Chestnut Hill, MA (US)

(73) Assignee: THE BRIGHAM AND WOMEN'S HOSPITAL, INC., BOSTON, MA (US)

(21) Appl. No.: 14/442,903

(22) PCT Filed: Nov. 15, 2013
(86) PCT No.: PCT/US13/70238

§ 371 Date: May 14, 2015

Related U.S. Application Data
(60) Provisional application No. 61/832,208, filed on Jun. 7, 2013.
(60) Provisional application No. 61/726,896, filed on Nov. 15, 2012.

(51) Int. Cl. G01N 33/68

(52) U.S. Cl. CPC G01N 33/689; G01N 2333/4716; G01N 2800/368

(57) ABSTRACT

Described herein are methods, systems and kits of the diagnosis of preeclampsia and HELLP syndrome, as well as the selection of a preeclampsia or HELLP syndrome treatment. These are based, at least in part, on the finding that proximal tubule injury, as measured by urinary KIM-1, is increased in severe preeclampsia and correlates with complement activation. The detection of complement proteins correlate with kidney injury in severe preeclampsia and/or HELLP syndrome and terminal complement blockage is a therapeutic approach taken as described in various embodiments of the present invention.

### CROSS-REFERENCE TO RELATED APPLICATIONS

This application claims benefit under 35 U.S.C. §119(e) of U.S. Provisional Application No. 61/726,896 filed Nov. 15, 2012 and of U.S. Provisional Application No. 61/832,208 filed Jun. 7, 2013, the contents of each of which are incorporated herein by reference in their entireties.

### BACKGROUND

Severe preeclampsia is associated with excessive and dysregulated terminal complement activation, yet the relationship between complement proteins and kidney injury in preeclampsia is poorly understood. Left untreated, preeclampsia can lead to serious, and even fatal, complications for both the woman and the fetus.

HELLP syndrome is a life-threatening liver disorder thought to be a type of severe preeclampsia. It is characterized by hemolysis (destruction of red blood cells), elevated liver enzymes (which indicate liver damage), and low platelet count. HELLP is usually related to preeclampsia. HELLP syndrome often occurs without warning and can be difficult to recognize. It can occur without the signs of preeclampsia (for example, a large increase in blood pressure and protein in the urine).

Accordingly, there exists a need in the art for methods, systems and kits to diagnose and treat preeclampsia and HELLP syndrome.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of diagnosing preeclampsia or HELLP syndrome, optionally selecting a preeclampsia therapy or HELLP syndrome therapy and optionally administering the preeclampsia therapy or HELLP syndrome therapy, comprising: assaying a biological sample obtained from a subject to determine the level of a preeclampsia biomarker selected from the group consisting of kidney injury molecule 1 (KIM-1), a complement protein or component, and combinations thereof; comparing the preeclampsia biomarker level to a reference level; and diagnosing preeclampsia if the level of the preeclampsia biomarker is higher than the reference level.

In various embodiments, the method can further comprise selecting a preeclampsia therapy or HELLP syndrome therapy if preeclampsia or HELLP syndrome is diagnosed.

In various embodiments, the method can further comprise administering the preeclampsia therapy or HELLP syndrome therapy to treat preeclampsia or HELLP syndrome if preeclampsia or HELLP syndrome is diagnosed.

In various embodiments, the complement protein or component can be C3a, C5a, C5b-9 or combinations thereof. In various embodiments, the complement protein or component can be a complement protein or component upstream from KIM-1. In various embodiments, the complement protein or component can be a complement protein or component downstream from KIM-1.

In various embodiments, the biological sample can be urine and the level of the preeclampsia biomarker can be normalized to urine creatinine level.

In various embodiments, the preeclampsia therapy or HELLP syndrome therapy can comprise a complement protein inhibitor. In various embodiments, the preeclampsia therapy or HELLP syndrome therapy can comprise Eculizumab. In various embodiments, the preeclampsia therapy or HELLP syndrome therapy can comprise Pexelizumab. In various embodiments, the preeclampsia therapy or HELLP syndrome therapy can comprise Compstatin. In various embodiments, the preeclampsia therapy or HELLP syndrome therapy can comprise sCR1. In various embodiments, the preeclampsia therapy or HELLP syndrome therapy can comprise Heparin. In various embodiments, the preeclampsia therapy or HELLP syndrome therapy can comprise magnesium sulfate, an antihypertensive drug, or combinations thereof.

In various embodiments, the preeclampsia therapy or HELLP syndrome therapy can comprise hydralazine, labetalol, nifedipine, sodium nitroprusside or a combination thereof.

In various embodiments, the method can further comprise assaying the biological sample to determine the level of an additional preeclampsia biomarker selected from the group consisting of PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, BNP, and combinations thereof; and comparing the level of the additional preeclampsia biomarker to a reference level for the additional preeclampsia biomarker; and wherein diagnosing preeclampsia or HELLP syndrome comprises diagnosing preeclampsia if the level of the preeclampsia biomarker is higher than the reference level and if the level of the additional preeclampsia biomarker differs from the reference level for the additional preeclampsia biomarker.

In various embodiments, the method can further comprise assaying the biological sample to determine the level of an additional preeclampsia biomarker selected from the group consisting of PlGF, PP13, PAPP-A, Inhibin A, Activin A, MMP-9, Endothelin-1, P-selectin, NGAL, sEng, triglycerides, HDL cholesterol, LDL cholesterol, and combinations thereof.

In various embodiments, the method can further comprise diagnosing preeclampsia or HELLP syndrome if the level of the preeclampsia biomarker is higher than the reference level, and if the preeclampsia biomarker is selected from the group consisting of PlGF, PP13, PAPP-A, HDL cholesterol, and combinations thereof, and the level of the preeclampsia biomarker is lower than the reference level, or if the preeclampsia biomarker is selected from the group consisting of Inhibin A, Activin A, MMP-9, Endothelin-1, P-selectin, sEng, NGAL, triglycerides, LDL cholesterol and combinations thereof, and the level of the preeclampsia biomarker is higher than the reference level.

Various embodiments of the present invention provide for a system for diagnosing preeclampsia or HELLP syndrome in a subject in need thereof, comprising: a sample analyzer configured to produce a signal for a preeclampsia biomarker a biological sample of a the subject; and a computer sub-system programmed to calculate, based on the preeclampsia biomarker whether the signal is higher than a reference value.

Various embodiments of the present invention provide for a computer program product embodied in a non-transitory computer readable medium that, when executing on a computer, performs steps comprising: detecting the preeclampsia biomarker level in a biological sample from a subject in need of a diagnosis regarding preeclampsia; and comparing the preeclampsia biomarker level to a reference level.

Various embodiments of the present invention provide for a kit for diagnosing preeclampsia or HELLP syndrome in a subject in need thereof, comprising: one or more probes comprising a combination of detectably labeled probes for the detection of KIM-1, one or more complement proteins or components, or combinations thereof. In various embodiments, the one or more complement proteins or components can be C3a, C5a, or C5b-9.

In various embodiments, the kit can further comprise the computer program product embodied in a non-transitory computer readable medium that, when executing on a computer, performs steps comprising: detecting the preeclampsia biomarker level in a biological sample from a subject in need of a diagnosis regarding preeclampsia or HELLP syndrome; and comparing the preeclampsia biomarker level to a reference level.

Various embodiments of the present invention provide for method of selecting a preeclampsia therapy or HELLP syndrome therapy, and optionally administering the preeclampsia therapy, comprising: assaying a biological sample obtained from a subject to determine the level of a preeclampsia biomarker; comparing the preeclampsia biomarker level to a reference level; and diagnosing preeclampsia if the level of the preeclampsia biomarker differs from the reference level; and selecting a preeclampsia therapy or HELLP syndrome therapy comprising a complement protein inhibitor if preeclampsia is diagnosed.

In various embodiments, the preeclampsia biomarker can be selected from the group consisting of PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, BNP, and combinations thereof.

In various embodiments, the method can further comprise administering the preeclampsia therapy or HELLP syndrome therapy comprising the complement protein inhibitor.

In various embodiments, the complement protein inhibitor can be Eculizumab. In various embodiments, the complement protein inhibitor can be Pexelizumab. In various embodiments, the complement protein inhibitor can be Compstatin. In various embodiments, the complement protein inhibitor can be sCR1. In various embodiments, the complement protein inhibitor can be Heparin.

In various embodiments, the preeclampsia therapy or HELLP syndrome therapy can further comprise magnesium sulfate, an antihypertensive drug, or combinations thereof. In various embodiments, the preeclampsia therapy or HELLP syndrome therapy can further comprise hydralazine, labetalol, nifedipine, sodium nitroprusside or a combination thereof.

Various embodiments of the present invention provide for a method of selecting a preeclampsia therapy or HELLP syndrome therapy, and optionally administering the preeclampsia therapy or HELLP syndrome therapy, comprising: assaying a biological sample obtained from a subject to determine the level of a preeclampsia biomarker selected from the group consisting of PlGF, PP13, PAPP-A, Inhibin A, Activin A, MMP-9, Endothelin-1, P-selectin, sEng, NGAL, triglycerides, HDL cholesterol, LDL cholesterol, and combinations thereof; comparing the preeclampsia biomarker level to a reference level; diagnosing preeclampsia or HELLP syndrome if the preeclampsia biomarker is selected from the group consisting of PlGF, PP13, PAPP-A, HDL cholesterol, and combinations thereof, and the level of the preeclampsia biomarker is lower than the reference level, or diagnosing preeclampsia or HELLP syndrome if the preeclampsia biomarker is selected from the group consisting of Inhibin A, Activin A, MMP-9, Endothelin-1, P-selectin, sEng, NGAL, triglycerides, LDL cholesterol and combinations thereof, and the level of the preeclampsia biomarker is higher than the reference level; and selecting a preeclampsia therapy or HELLP syndrome therapy comprising a complement protein inhibitor if preeclampsia is diagnosed.

In various embodiments, the method can further comprise administering the preeclampsia therapy or HELLP syndrome therapy comprising the complement protein inhibitor.

In various embodiments, the complement protein inhibitor can be Eculizumab. In various embodiments, the complement protein inhibitor can be Pexelizumab. In various embodiments, the complement protein inhibitor can be Compstatin. In various embodiments, the complement protein inhibitor can be sCR1. In various embodiments, the complement protein inhibitor can be Heparin.

In various embodiments, the preeclampsia therapy or HELLP syndrome therapy can further comprise magnesium sulfate, an antihypertensive drug, or combinations thereof. In various embodiments, the preeclampsia therapy or HELLP syndrome therapy can further comprise hydralazine, labetalol, nifedipine, sodium nitroprusside or a combination thereof.

Various embodiments of the present invention provide for an assay for diagnosing preeclampsia or HELLP syndrome, comprising: a solid phase; a first reagent to react with a preeclampsia biomarker, wherein the first reagent is immobilized on the solid phase; a second reagent to specifically react with a preeclampsia biomarker, wherein the second reagent comprises a label; a substrate to react with the label; a third reagent to react with the second reagent to serve as a control. In various embodiments, the solid phase can be a capillary membrane.

Various embodiments of the present invention provide for a method of diagnosing preeclampsia or HELLP syndrome, comprising: contacting a urine sample from a subject in need of a diagnosis regarding preeclampsia or HELLP syndrome to an assay of the present invention, wherein two marks indicate a diagnosis of preeclampsia or HELLP syndrome and one mark indicates a negative result.

Various embodiments of the present invention provide for a method of treating preeclampsia or HELLP syndrome, comprising: administering a complement protein inhibitor to a subject in need thereof. In various embodiments, the complement protein inhibitor can be Eculizumab. In various embodiments, the complement protein inhibitor can be Pexelizumab. In various embodiments, the complement protein inhibitor can be Compstatin. In various embodiments, the complement protein inhibitor can be sCR1. In various embodiments, the complement protein inhibitor can be Heparin.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts characteristics of subjects with severe preeclampsia. Fetal growth restriction, less than 10th percentile for gestational age. AST, aspartate transaminase. Reference range (10-50 U/L). HELLP, hemolysis, elevated liver enzymes, low platelets; Sibai Criteria²³

FIG. 2A depicts U—C3a/U—Cr ratio in severe preeclampsia vs. controls.

FIG. 2B depicts U—C5a/U—Cr ratio in severe preeclampsia vs. controls.

FIG. 2C depicts Urinary concentrations of C3a, C5a, C5b-9 and PlGF in healthy controls, chronic hypertension and severe preeclampsia. Median (horizontal line), interquartile range (box), range (whiskers), outliers >1.5 SD from upper quartile excluded for display purposes. * C3a (ng/dl)⁻¹, C5a (ng/dl), C5b-9 (ng/dl)⁻¹, PlGF (ng/dl); Concentrations varied for display purposes; + p<0.01, preeclampsia vs. healthy controls or chronic hypertension; ∘ p<0.01, preeclampsia or chronic hypertension vs. healthy controls; • p<0.0001, preeclampsia vs. healthy controls or chronic hypertension

FIG. 3 depicts scatter plots of plasma C3a and C5a levels, stratified by group.

FIG. 4 depicts plasma and urine C5b-9 levels in relation to Eculizumab treatment in preeclampsia.

FIGS. 5A and 5B depict lab trends in relation to Eculizumab. a. LDH and haptoglobin; b. AST, ALT and platelet count.

FIG. 6 depicts urinary KIM-1 adjusted for creatinine in cases and controls. Median (horizontal line), interquartile range (box), range (whiskers), outliers >1.5 SD from upper quartile (dots). + p<0.0001, preeclampsia vs. CHTN or healthy controls.

FIG. 7 shows that the complement cascade may be activated through classical (CP), lectin (LP), or alternative pathways (AP). While each pathway has distinct triggers (e.g., immune complexes, CP), they all converge to generate C3 convertases, specifically C4b2b (CP, LP) and C3bBb (AP). C3 convertases cleave C3 to generate complement activation products C3a (anaphylatoxin) and C3b (opsonin). Activated C3b also contributes to generation of C5 convertases, specifically C4b2a3b (CP, LP) and C3bBb3b (AP). C5 convertases cleave C5 to generate C5a (anaphylatoxin) and C5b, which combines with complement proteins C6-9 to form C5b-9 (membrane attack complex).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described.

Central to innate immunity, complement activation is heightened in pregnancy¹ in part to facilitate normal clearance of feto-placental material including apoptotic blebs², circulating fetal DNA³, and immune complexes⁴. However, in severe preeclampsia, the burden of this debris is exaggerated⁵⁻⁹ and complement signaling becomes dysregulated¹⁰⁻¹³. Excessive generation of C5a and C5b-9 propagates endothelial injury¹⁴⁻¹⁵, angiogenic dysregulation^{16,17}, and triggers a systemic inflammatory response that manifests clinically as preeclampsia^{10,8-20}.

The kidney is at particular risk of complement-mediated injury.¹⁵ C5a-induced inflammatory responses contribute to renal tissue destruction and C5b-9 mediates direct renal tubular injury.²¹⁻²² Considering that kidney injury is fundamental to the diagnosis and pathophysiology of preeclampsia, and while not wishing to be bound by any particular theory, we believe that urinary complement levels, more so than plasma levels, are exaggerated in severe disease.

In severe preeclampsia heightened activation of downstream complement protein C5 leads to excess generation of C5a and C5b-9 (13-16). C5a propagates a potent pro-inflammatory response (13, 24, 25-26), while C5b-9 incorporates into cell membranes, including villous trophoblast (27), and contributes to platelet activation, procoagulant effects, and lytic cell death (28-31). Additionally, C5a stimulates monocytes to release soluble fms-like tyrosine kinase 1 (sFLT-1) (32) which sequesters vascular endothelial growth factor (VEGF) and placental growth factor (PlGF), contributing to hypertension and glomerular endotheliosis (33-34).

Our results introduce the novel finding that complement activation products C3a, C5a, and C5b-9 are excreted in the urine in association with severe preeclampsia. While urinary excretion of C3a, C5a and C5b-9 was exaggerated in severe preeclampsia compared to healthy controls, excretion of C5b-9 distinguished most clearly between severe preeclampsia and chronic hypertension. As a biomarker of disease urinary C5b-9 was superior to plasma C5b-9, which could not distinguish between cases and hypertensive controls, supporting our hypothesis that complement markers in urine rather than plasma better reflect complement dysregulation.

Considering that plasma levels of C5a and C5b-9 were increased in subjects with preeclampsia, it is possible that urinary excretion of C5a and C5b-9 occurred because normal plasma clearance mechanisms (35) were overwhelmed. However, we found that urinary complement levels (C3a, C5a or C5b-9) did not correlate with plasma complement levels among women with severe preeclampsia, arguing against simple renal clearance of circulating complement proteins. While urinary excretion of complement proteins correlate with blood pressure and proteinuria, among cases of preeclampsia urinary complement markers correlated more strongly with each other than with markers of renal impairment. We hypothesize that exaggerated and coordinated urinary excretion of C3a, C5a and C5b-9 in severe preeclampsia occurred due to complement-mediated inflammation and injury in the kidney, possibly at the level of the proximal tubule as seen in renal ischemia reperfusion injury (17-18).

While urinary levels of C3a, C5a and C5b-9 were all increased in severe preeclampsia, downstream markers of C5 activation, particularly C5b-9, were more exaggerated in disease. Similarly, plasma levels of C5a and C5b-9, but not C3a, were increased in severe preeclampsia and chronic hypertension. Increased plasma C5 activation in chronic hypertensives may reflect heightened endothelial dysfunction or systemic inflammation and is consistent with the general finding that chronic hypertension in pregnancy predisposes to severe preeclampsia. (36)

Taken together, our data suggest that complement dysregulation in ongoing disease occurs primarily at the level of C5. Animal models also support a dominant role for C5, relative to C3, in both complement-mediated kidney injury and adverse pregnancy outcomes (17-18, 32). Levels of C5a and C5b-9 may rise out of proportion to C3a because of direct C5 activation (i.e., enzymatic cleavage) by extrinsic serine proteases, bypassing proximal complement pathways (37-38). These extrinsic activators of C5 may be derived from leukocytes or the coagulation cascade (e.g., thrombin (37)). Alternatively, complement gene mutations (e.g., CD46 gene mutations (39)), may predispose to increased production of C5 convertases that generate C5a and C5b.

Presumably, C5a and C5b-9 should be generated in similar amounts from C5 activation. However, C5a has very high affinity for its receptors and is rapidly cleared from circulation (35). Furthermore, distinct C5a receptors in the kidney may be expressed in the proximal tubule or thick ascending limb of Henle's loop (CSaR) or the distal convoluting tubule (C5L2) (40-42), which may limit the usefulness of urinary C5a as a biomarker of disease activity. C5b-9 appears to be a more reliable urinary marker for various renal diseases (19-20). C5b-9 may be formed at the glomerular membrane and shed into the urine (43) or complement proteins may pass through the glomerular membrane into the tubular lumen to stimulate complement activation and formation of C5b-9 in the proximal tubule (44). Furthermore, our data also shows that increased levels of urinary C5b-9 but not C3a or C5a correlates well (i.e., inversely) with urinary PlGF, a validated marker of the altered angiogenic state in preeclampsia. (45)

Our findings support the concept that severe preeclampsia is propagated by excess complement activation and eventual complement dysregulation, particularly at the level of C5. We propose that during gestation increasing amounts of aponecrotic feto-placental debris (6, 9-10) secondary to early placental aberrations, underlying co-morbid conditions (e.g., chronic hypertension) or genetic factors, may trigger excess complement activation which propagates systemic inflammation (13, 24, 46-47), angiogenic imbalance (32), endothelial dysfunction (48-50), coagulation activation (26, 28-29, 37) and oxidative stress (50-51), with eventual kidney injury (17-18, 33-34) and hypertension (33-34). While not wishing to be bound by any particular theory, we believe that complement-mediated inflammation and injury in the kidney is particularly detrimental in severe preeclampsia as suggested by marked urinary excretion of C5b-9 during active disease. While heightened C5 activation occurred in pregnant gravidas with chronic hypertension, urinary excretion of C5b-9 was specific to preeclampsia. Our findings support complement proteins, for example, urinary C5b-9, as a biomarker for severe preeclampsia, which may distinguish pathologic complement dysregulation from simply heightened complement activation. Furthermore, our finding that complement dysregulation is common in severe preeclampsia suggests that complement inhibition may be a viable treatment option.

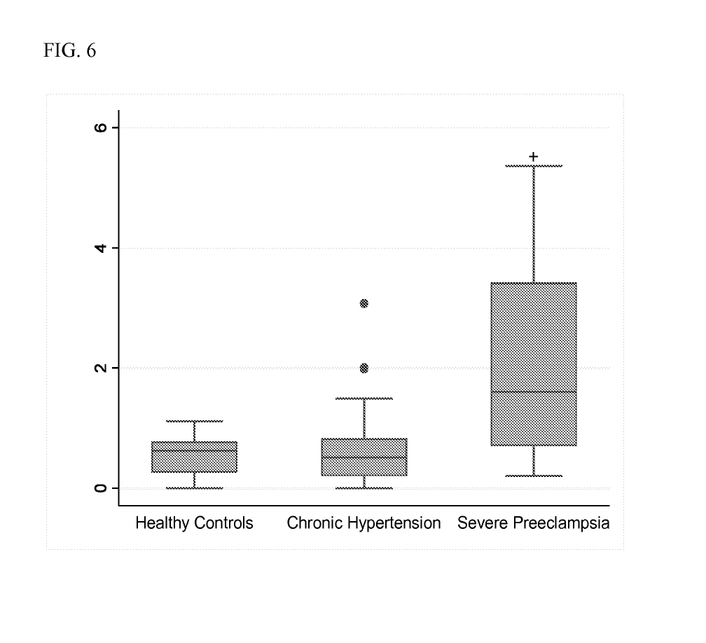

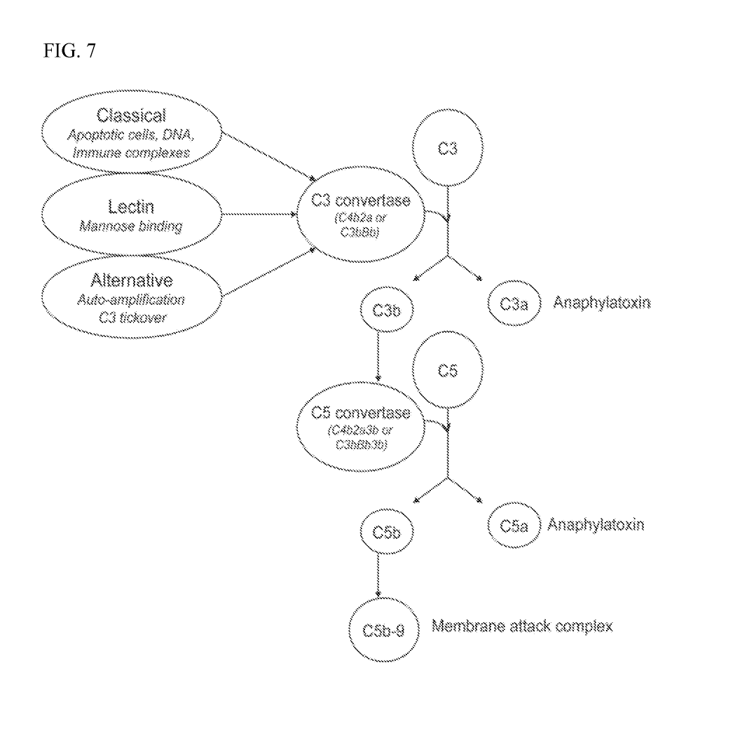

Proximal tubule injury, as measured by urinary KIM-1, is increased in severe preeclampsia and correlates with complement activation. Downstream, rather than upstream, complement proteins correlate with kidney injury in severe preeclampsia suggesting that terminal complement blockade may have some therapeutic rationale. (See FIG. 6.)

Our findings support our belief that complement dysregulation, particularly excessive C5 activation, is central to ongoing disease in severe preeclampsia and it remains an intriguing pathway for targeted therapies. Accordingly, various embodiments of the present invention are based, at least in part, upon these findings.

### Diagnosis

Described herein, method, systems and kits for diagnosing preeclampsia and HELLP syndrome are provided based on the detection of preeclampsia biomarkers.

Various embodiments of the present invention provide for a method of diagnosing preeclampsia, comprising: assaying a biological sample obtained from a subject to determine the level of a preeclampsia biomarker selected from the group consisting of kidney injury molecule 1 (KIM-1), a complement protein or component, and combinations thereof; comparing the preeclampsia biomarker level to a reference level; and diagnosing preeclampsia if the level of the preeclampsia biomarker is higher than the reference level. In various embodiments, the method further comprises selecting a preeclampsia therapy if preeclampsia is diagnosed. In various embodiments, the method further comprises administering the preeclampsia therapy to treat preeclampsia if preeclampsia is diagnosed. Preeclampsia therapies that can be selected and/or administered are further described herein.

Various embodiments of the present invention provide for a method of diagnosing HELLP syndrome, comprising: assaying a biological sample obtained from a subject to determine the level of a preeclampsia biomarker selected from the group consisting of kidney injury molecule 1 (KIM-1), a complement protein or component, and combinations thereof; comparing the preeclampsia biomarker level to a reference level; and diagnosing HELLP syndrome if the level of the preeclampsia biomarker is higher than the reference level. In various embodiments, the method further comprises selecting a HELLP syndrome therapy if HELLP syndrome is diagnosed. In various embodiments, the method further comprises administering the HELLP syndrome therapy to treat HELLP syndrome if HELLP syndrome is diagnosed. HELLP syndrome therapies that can be selected and/or administered are further described herein.

In some embodiments, where distinguishing between preeclampsia and HELLP syndrome is desired, one can examine additional symptoms or biomarkers of HELLP syndrome in order to arrive at the diagnosis. For example, symptoms such as, headaches, nausea and vomiting that continue to get worse, upper right abdominal pain or tenderness, fatigue or malaise, visual disturbances, high blood pressure, protein in urine, edema, severe headaches, bleeding, Hemolysis (Red blood cells, Abnormal peripheral smear, Lacatate dehydrogenase >600 U/L, Bilirubin >1.2 mg/dl), Elevated liver Enzyme levels (Serum aspartate amniotransferase >70 U/L, Lacatate dehydrogenase >600 U/L), and Low Platelets. Alternatively, additional symptoms of preeclampsia can be examined to distinguish between preeclampsia and HELLP syndrome. For example, symptoms such as high blood pressure (e.g., 140/90 millimeters of mercury (mm Hg) or greater, documented on two or more occasions), proteinuria, severe headaches, changes in vision, including temporary loss of vision, blurred vision or light sensitivity, upper abdominal pain, usually under your ribs on the right side, nausea or vomiting, dizziness, decreased urine output, sudden weight gain, typically more than 2 pounds (0.9 kilogram) a week, edema (particularly in face and hands).

In various embodiments, the complement protein or component being detected can be C3a, C5a or C5b-9. In other embodiments, the complement protein or component being detected can be Bb or C4d. In other embodiments, the complement protein or component being detected can be C5b, C6, C7, C8, or C9. In various embodiments, the complement protein or component is a complement protein or component upstream from KIM-1. In various embodiments, the complement protein or component is a complement protein or component downstream from KIM-1. In some embodiments, markers of complement activation can be detected. For example, an increase in sFLT-1, a decrease in VEGF, or a decrease in PlGF can indicate complement activation. Other examples of complement activation include, but are not limited to anti-angiogentic factors, cytokines and other inflammatory mediators.

In various embodiments, the method can further comprise assaying for the level of additional preeclampsia biomarkers.

In various embodiments, the additional preeclampsia biomarkers are complement regulatory proteins. In certain embodiments, the complement regulatory proteins are CD46, CD55, and CD59.

In these embodiments, the method can further comprise assaying the biological sample to determine the level of an additional preeclampsia biomarker selected from the group consisting of CD46, CD55, and CD59, and combinations thereof; and comparing the level of the additional preeclampsia biomarker to a reference level for the additional preeclampsia biomarker; and wherein diagnosing preeclampsia or HELLP syndrome comprises diagnosing preeclampsia or HELLP syndrome if the level of the preeclampsia biomarker is higher than the reference level and if the level of the additional preeclampsia biomarker differs from the reference level for the additional preeclampsia biomarker.

Other preeclampsia biomarkers are known in the art. (See e.g., Griffin and Chappell, Prediction of Pre-eclampsia—Screening, Biomarkers and Perspectives for the Future, EUROPEAN OBSTETRICS & GYNAECOLOGY, 2012; 7(1):18-24.) Examples of additional preeclampsia biomarkers include, but are not limited to PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, and BNP.

In these embodiments, the method can further comprise assaying the biological sample to determine the level of an additional preeclampsia biomarker selected from the group consisting of PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, BNP, and combinations thereof; and comparing the level of the additional preeclampsia biomarker to a reference level for the additional preeclampsia biomarker; and wherein diagnosing preeclampsia or HELLP syndrome comprises diagnosing preeclampsia or HELLP syndrome if the level of the preeclampsia biomarker is higher than the reference level and if the level of the additional preeclampsia biomarker differs from the reference level for the additional preeclampsia biomarker.

In these embodiments, the method can further comprise diagnosing preeclampsia or HELLP syndrome if the additional preeclampsia biomarker is selected from the group consisting of PlGF, PP13, PAPP-A, HDL cholesterol, and combinations thereof, and the level of the additional preeclampsia biomarker is lower than the reference level, or diagnosing preeclampsia if the additional preeclampsia biomarker is selected from the group consisting of Inhibin A, Activin A, MMP-9, Endothelin-1, P-selectin, sEng, NGAL, triglycerides, LDL cholesterol and combinations thereof, and the level of the additional preeclampsia biomarker is higher than the reference level.

Various embodiments of the present invention provide for a system for diagnosing preeclampsia, comprising: a biological sample obtained from a subject who desires a diagnosis regarding preeclampsia; and an assay to determine the level of a preeclampsia biomarker selected from the group consisting of kidney injury molecule 1 (KIM-1), a complement protein or component, and combinations thereof.

Various embodiments of the present invention provide for a system for diagnosing HELLP syndrome, comprising: a biological sample obtained from a subject who desires a diagnosis regarding HELLP syndrome; and an assay to determine the level of a preeclampsia biomarker selected from the group consisting of kidney injury molecule 1 (KIM-1), a complement protein or component, and combinations thereof.

In various embodiments, the complement protein or component being detected can be C3a, C5a or C5b-9. In other embodiments, the complement protein or component being detected can be Bb or C4d. In other embodiments, the complement protein or component being detected can be C5b, C6, C7, C8, or C9. In various embodiments, the complement protein or component is a complement protein or component upstream from KIM-1. In various embodiments, the complement protein or component is a complement protein or component downstream from KIM-1. In some embodiments, markers of complement activation can be detected. For example, an increase in sFLT-1, a decrease in VEGF, or a decrease in PlGF can indicate complement activation. Other examples of complement activation include, but are not limited to anti-angiogentic factors, cytokines and other inflammatory mediators.

In various embodiments, the system can further comprise assays to determine the level of additional preeclampsia biomarkers. Examples of additional preeclampsia biomarkers include, but are not limited to CD46, CD55, CD59, PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, and BNP.

Various embodiments of the present invention provide for a system for diagnosing preeclampsia in a subject in need thereof, comprising: a sample analyzer configured to produce a signal for a preeclampsia biomarker in a biological sample of the subject; and a computer sub-system programmed to calculate, based on the preeclampsia biomarker whether the signal is higher than a reference value. In various embodiments, the system further comprises the biological sample.

Various embodiments of the present invention provide for a system for diagnosing HELLP syndrome in a subject in need thereof, comprising: a sample analyzer configured to produce a signal for a preeclampsia biomarker in a biological sample of the subject; and a computer sub-system programmed to calculate, based on the preeclampsia biomarker whether the signal is higher than a reference value. In various embodiments, the system further comprises the biological sample.

Various embodiments of the present invention provide for a computer program product embodied in a non-transitory computer readable medium that, when executing on a computer, performs steps comprising: detecting the preeclampsia biomarker level in a biological sample from a subject in need of a diagnosis regarding preeclampsia; and comparing the preeclampsia biomarker level to a reference level.

Various embodiments of the present invention provide for a computer program product embodied in a non-transitory computer readable medium that, when executing on a computer, performs steps comprising: detecting the preeclampsia biomarker level in a biological sample from a subject in need of a diagnosis regarding HELLP syndrome; and comparing the preeclampsia biomarker level to a reference level.

The present invention is also directed to a kit to diagnose or treat preeclampsia or HELLP syndrome. The kit is an assemblage of materials or components, including at least one of the inventive compositions or assays. The exact nature of the components configured in the inventive kit depends on its intended purpose. Instructions for use may be included in the kit. “Instructions for use” typically include a tangible expression describing the technique to be employed in using the components of the kit to diagnose or treat preeclampsia or HELLP syndrome. Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in point of care kits. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

Various embodiments of the present invention provide for a kit for diagnosing preeclampsia or HELLP syndrome in a subject in need thereof, comprising: one or more probes comprising a combination of detectably labeled probes for the detection of KIM-1, one or more complement proteins or components, or a combination thereof. In various embodiments, the one or more complement proteins or components are C3a, C5a, or C5b-9. In other embodiments, the complement protein or component can be Bb or C4d. In other embodiments, the complement protein or component being detected can be C5b, C6, C7, C8, or C9. In still other embodiments, the kit can further comprise one or more probes comprising a combination of detectably labeled probes for the detection of complement regulatory proteins such as CD46, CD55, and CD59. In various embodiments, the kit further comprises the computer program product embodied in a non-transitory computer readable medium that, when executing on a computer, performs steps comprising: detecting the preeclampsia biomarker level in a biological sample from a subject in need of a diagnosis regarding preeclampsia or HELLP syndrome; and comparing the preeclampsia biomarker level to a reference level.

In various embodiments, the kit comprises an assay to detect the levels of the preeclampsia biomarker; for example, levels of KIM-1, complement protein or component C3a, C5a, or C5b-9, complement regulatory proteins (e.g., CD46, CD55, and CD59). In various embodiments, the assay comprises a control (e.g., reference level for comparison to the test level).

In various embodiments the kit comprises an assay as discussed herein and instructions to use the assay to diagnose preeclampsia.

### Selecting Therapy

Described herein, methods, systems and kits for selecting a therapy to treat preeclampsia or HELLP syndrome are provided based on the detection of preeclampsia biomarkers and therapeutic effects of complement protein inhibitors.

Various embodiments of the present invention provide for a method of selecting a preeclampsia therapy, comprising: assaying a biological sample obtained from a subject to determine the level of a preeclampsia biomarker selected from the group consisting of kidney injury molecule 1 (KIM-1), a complement protein or component, and combinations thereof; comparing the preeclampsia biomarker level to a reference level; diagnosing preeclampsia if the level of the preeclampsia biomarker is higher than the reference level; and selecting a preeclampsia therapy if preeclampsia is diagnosed.

Various embodiments of the present invention provide for a method of selecting a HELLP syndrome therapy, comprising: assaying a biological sample obtained from a subject to determine the level of a preeclampsia biomarker selected from the group consisting of kidney injury molecule 1 (KIM-1), a complement protein or component, and combinations thereof; comparing the preeclampsia biomarker level to a reference level; diagnosing HELLP syndrome if the level of the preeclampsia biomarker is higher than the reference level; and selecting a HELLP syndrome therapy if preeclampsia is diagnosed.

In various embodiments, the complement protein or component being detected can be C3a, C5a or C5b-9. In other embodiments, the complement protein or component being detected can be Bb or C4d. In other embodiments, the complement protein or component being detected can be C5b, C6, C7, C8, or C9. In various embodiments, the complement protein or component is a complement protein or component downstream from KIM-1. In some embodiments, the complement protein or component is a complement protein or component upstream from KIM-1. In some embodiments, markers of complement activation can be detected. For example, an increase in sFLT-1, a decrease in VEGF, or a decrease in PlGF can indicate complement activation. Other examples of complement activation include, but are not limited to anti-angiogentic factors, cytokines and other inflammatory mediators.

In various embodiments, the method further comprises administering the preeclampsia therapy or HELLP syndrome to treat preeclampsia or HELLP syndrome if preeclampsia or HELLP syndrome is diagnosed. Preeclampsia therapies and HELLP syndrome therapies that can be selected and/or administered are further described herein.

In various embodiments, the method can further comprising assaying for the level of additional preeclampsia biomarkers.

In various embodiments, the additional preeclampsia biomarkers are complement regulatory proteins. In certain embodiments, the complement regulatory proteins are CD46, CD55, and CD59.

In these embodiments, the method can further comprise assaying the biological sample to determine the level of an additional preeclampsia biomarker selected from the group consisting of CD46, CD55, and CD59, and combinations thereof; and comparing the level of the additional preeclampsia biomarker to a reference level for the additional preeclampsia biomarker; and wherein diagnosing preeclampsia or HELLP syndrome comprises diagnosing preeclampsia or HELLP syndrome if the level of the preeclampsia biomarker is higher than the reference level and if the level of the additional preeclampsia biomarker differs from the reference level for the additional preeclampsia biomarker.

Other preeclampsia biomarkers are known in the art. (See e.g., Griffin and Chappell, Prediction of Pre-eclampsia—Screening, Biomarkers and Perspectives for the Future, EUROPEAN OBSTETRICS & GYNAECOLOGY, 2012; 7(1):18-24.) Examples of additional preeclampsia biomarkers include, but are not limited to PlGF, PP13, PAPP-A, Inhibin A, Activin A, MMP-9, Endothelin-1, P-selectin, NGAL, sEng, triglycerides, HDL cholesterol, LDL cholesterol. In these embodiments, the method can further comprise diagnosing preeclampsia or HELLP syndrome if the additional preeclampsia biomarker is selected from the group consisting of PlGF, PP13, PAPP-A, HDL cholesterol, and combinations thereof, and the level of the additional preeclampsia biomarker is lower than the reference level, or diagnosing preeclampsia if the additional preeclampsia biomarker is selected from the group consisting of Inhibin A, Activin A, MMP-9, Endothelin-1, P-selectin, sEng, NGAL, triglycerides, LDL cholesterol and combinations thereof, and the level of the additional preeclampsia biomarker is higher than the reference level.

Based on our findings, described herein are also methods of selecting a preeclampsia therapy or a HELLP syndrome therapy based on known preeclampsia biomarkers. Thus, various embodiments of the present invention provide for a method of selecting a preeclampsia therapy or HELLP syndrome therapy, and optionally administering the preeclampsia therapy or HELLP syndrome therapy, comprising: assaying a biological sample obtained from a subject to determine the level of a preeclampsia biomarker selected from the group consisting of PlGF, PP13, PAPP-A, Inhibin A, Activin A, MMP-9, Endothelin-1, P-selectin, sEng, NGAL, triglycerides, HDL cholesterol, LDL cholesterol, and combinations thereof; comparing the preeclampsia biomarker level to a reference level; diagnosing preeclampsia if preeclampsia biomarker is selected from the group consisting of PlGF, PP13, PAPP-A, HDL cholesterol, and combinations thereof, and the level of the preeclampsia biomarker is lower than the reference level, or diagnosing preeclampsia if the preeclampsia biomarker is selected from the group consisting of Inhibin A, Activin A, MMP-9, Endothelin-1, P-selectin, sEng, NGAL, triglycerides, LDL cholesterol and combinations thereof, and the level of the preeclampsia biomarker is higher than the reference level; and selecting a preeclampsia or HELLP syndrome therapy comprising a complement protein inhibitor if preeclampsia is diagnosed.

Other embodiments of the present invention provide for a method of selecting a HELLP syndrome therapy, and optionally administering the HELLP syndrome therapy, comprising: assaying a biological sample obtained from a subject to determine the level of a preeclampsia biomarker selected from the group consisting of PlGF, PP13, PAPP-A, Inhibin A, Activin A, MMP-9, Endothelin-1, P-selectin, sEng, NGAL, triglycerides, HDL cholesterol, LDL cholesterol, and combinations thereof; comparing the preeclampsia biomarker level to a reference level; diagnosing preeclampsia if preeclampsia biomarker is selected from the group consisting of PlGF, PP13, PAPP-A, HDL cholesterol, and combinations thereof, and the level of the preeclampsia biomarker is lower than the reference level, or diagnosing preeclampsia if the preeclampsia biomarker is selected from the group consisting of Inhibin A, Activin A, MMP-9, Endothelin-1, P-selectin, sEng, NGAL, triglycerides, LDL cholesterol and combinations thereof, and the level of the preeclampsia biomarker is higher than the reference level; and selecting a HELLP syndrome therapy comprising a complement protein inhibitor if HELLP syndrome is diagnosed.

In various embodiments, the method can further comprise administering the preeclampsia therapy or HELLP syndrome therapy. Preeclampsia therapies and HELLP syndrome therapies that can be selected and/or administered are further described herein.

Various embodiments provide for a method of selecting a preeclampsia therapy, and optionally administering the preeclampsia therapy, comprising: assaying a biological sample obtained from a subject to determine the level of a preeclampsia biomarker; comparing the preeclampsia biomarker level to a reference level; diagnosing preeclampsia if the level of the preeclampsia biomarker differs from the reference level; and selecting a preeclampsia therapy comprising a complement protein inhibitor if preeclampsia is diagnosed. Examples of preeclampsia biomarkers include but are not limited to CD46, CD55, and CD59, PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1 and -R2, Complement activation product Bb, Triglycerides, PAI-2, IGFBP-1, Free foetal DNA, sFlt-1, sEng, BNP, and combinations thereof.

Other embodiments provide for a method of selecting a HELLP syndrome therapy, and optionally administering the HELLP syndrome therapy, comprising: assaying a biological sample obtained from a subject to determine the level of a preeclampsia biomarker; comparing the preeclampsia biomarker level to a reference level; diagnosing preeclampsia if the level of the preeclampsia biomarker differs from the reference level; and selecting a HELLP syndrome therapy comprising a complement protein inhibitor if preeclampsia is diagnosed. Examples of preeclampsia biomarkers include but are not limited to CD46, CD55, and CD59, PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1 and -R2, Complement activation product Bb, Triglycerides, PAI-2, IGFBP-1, Free foetal DNA, sFlt-1, sEng, BNP, and combinations thereof.

In various embodiments, the method further comprises administering the preeclampsia therapy or HELLP syndrome therapy.

### Assays

The assays used in the methods, systems and kits described herein can be assays known in the art. In various embodiments, the assays are assays as described herein.

Protein and Peptide Detection

One of ordinary skill in the art will readily appreciate methods and systems that can be used to detect the level of the preeclampsia biomarkers. These methods and systems include but are not limited to enzyme-linked immunosorbent assay (ELISA), immunohistochemistry, flow cytometry, fluorescence in situ hybridization (FISH), radioimmuno assays, and affinity purification. Examples of ELISAs include but are not limited to indirect ELISA, sandwich ELISA, competitive ELISA, multiple and portable ELISA.

In various embodiments of the methods, systems and kits described herein, the assay is an assay to detect the level of preeclampsia biomarkers. The assay can comprise: a first reagent (e.g., a capture antibody) to react with the preeclampsia biomarker in the biological sample if the biological sample comprises the preeclampsia biomarker (if preeclampsia biomarkers are not present, then the first reagent will not react with the preeclampsia biomarker in the biological sample, but the first reagent is still present in the assay), a second reagent (e.g., a detecting antibody) to react with the preeclampsia biomarkers, a third reagent (e.g., a secondary antibody) to react with the second reagent and a substrate (e.g., to react with a label on the third reagent and produce a signal). In various embodiments, the third reagent comprises a label to produce a signal to indicate the presence and/or level of the preeclampsia biomarker. In various embodiments, the label is a radiolabel, a chromophore, a fluorophore, a quantum dot, an enzyme, horseradish peroxidase (HRP), an alkaline phosphatase (AP), biotin, or a combination thereof. In various embodiments, the label is an enzyme that will react with the substrate. In various embodiments, the first reagent is on a solid phase (e.g., plate, multi-well plate).

In various embodiments, the first reagent, second reagent comprising a label, and substrate are all on one solid phase (e.g., dipstick). In a further embodiment, the first reagent, second reagent comprising a label, substrate, as well as control reagents, are all on one solid phase (e.g., dipstick).

In various embodiments, the assay comprises a solid phase; a first reagent to react with a preeclampsia biomarker, wherein the first reagent is immobilized on the solid phase; a second reagent to specifically react with a preeclampsia biomarker, wherein the second reagent comprise a label; a substrate to react with the label; a third reagent to react with the second reagent to serve as a control. In various embodiments, the solid phase is a capillary membrane. When used, two bands (circles or the like) can indicate a positive result (e.g., the patient has preeclampsia; and one band (circles or the like) can indicate a negative result. In various embodiments, the first reagent can be an antibody that specifically binds to the preeclampsia biomarker. In various embodiments, the control region or a further control region of the assay can be configured to produce a signal for comparing the test region to the control region such that one can determine if the test sample has a higher or lower level of the preeclampsia biomarker.

In various embodiments, the preeclampsia biomarker can be ones discussed herein (e.g., KIM-1, complement proteins or components, complement protein or component C3a, C5a, C5b-9, Bb, C4d, C5b, C6, C7, C8, or C9, complement regulatory proteins (e.g., CD46, CD55, and CD59), biomarkers discussed in Griffin and Chappell).

In various embodiments the substrate is a chromogenic substrate (e.g., 3,3′,5,5′-Tetramethylbenzidine (TMB), 3,3′-Diaminobenzidine (DAB), 2,2′-azino-bis(3-ethylbenzothiazoline-6-sulphonic acid) (ABTS). In various embodiments, the substrate is a chemiluminescence substrate (e.g., ECL).

In various embodiments, the assay to detect the level of preeclampsia biomarkers also comprises a control.

### Reference Levels

In various embodiments of the present invention, the reference level is based on the normal level or normal range of the same biomarker in subject who does not have preeclampsia. In various embodiments, the subject who does not have preeclampsia can be a pregnant woman, a pregnant woman in her first trimester, a pregnant woman in her second trimester or a pregnant woman in her third trimester.

In certain embodiments, depending on the preeclampsia biomarker, a preeclampsia biomarker level higher than a reference level is indicative of preeclampsia. In certain embodiments, depending on the preeclampsia biomarker, a preeclampsia biomarker level higher than a reference level is indicative of preeclampsia. In these embodiments, preeclampsia biomarker level can be increased by at least or about 5, 10, 20, 30, 40, 50, 60, 70, 80, or 90% compared to reference level to result in a diagnosis of preeclampsia. In various embodiments, the preeclampsia biomarker level can be increased by at least or about 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold 2.2-fold 2.3-fold 2.4-fold 2.5-fold, 2.6-fold, 2.7-fold, 2.8-fold, 2.9-fold, or 3-fold, 4-fold, 5-fold, 6-fold, 7-fold, 8-fold, 9-fold or 10-fold compared to reference level.

In certain embodiments, depending on the preeclampsia biomarker, a preeclampsia biomarker level higher than a reference level is indicative of HELLP syndrome. In certain embodiments, depending on the preeclampsia biomarker, a preeclampsia biomarker level higher than a reference level is indicative of HELLP syndrome. In these embodiments, preeclampsia biomarker level can be increased by at least or about 5, 10, 20, 30, 40, 50, 60, 70, 80, or 90% compared to reference level to result in a diagnosis of HELLP syndrome. In various embodiments, the preeclampsia biomarker level can be increased by at least or about 1-fold, 1.1-fold, 1.2-fold, 1.3-fold, 1.4-fold, 1.5-fold, 1.6-fold, 1.7-fold, 1.8-fold, 1.9-fold, 2-fold, 2.1-fold 2.2-fold 2.3-fold 2.4-fold 2.5-fold, 2.6-fold, 2.7-fold, 2.8-fold, 2.9-fold, or 3-fold, 4-fold, 5-fold, 6-fold, 7-fold, 8-fold, 9-fold or 10-fold compared to reference level.

In certain embodiments, depending on the preeclampsia biomarker, a preeclampsia biomarker level lower than a reference level is indicative of preeclampsia. In these embodiments, preeclampsia biomarker level can be decreased by at least or about 5, 10, 20, 30, 40, 50, 60, 70, 80, 90, 95, 96, 97, 98, or 99% compared to reference level to result in a diagnosis of preeclampsia.

In certain embodiments, depending on the preeclampsia biomarker, a preeclampsia biomarker level lower than a reference level is indicative of HELLP syndrome. In these embodiments, preeclampsia biomarker level can be decreased by at least or about 5, 10, 20, 30, 40, 50, 60, 70, 80, 90, 95, 96, 97, 98, or 99% compared to reference level to result in a diagnosis of HELLP syndrome.

In various embodiments, the reference level for a preeclampsia biomarker is the level for the same biomarker in a healthy subject (e.g., one who does not have preeclampsia, a pregnant woman who does not have preeclampsia, a pregnant woman in the same trimester who does not have preeclampsia, one who does not have HELLP syndrome, a pregnant woman who does not have HELLP syndrome, a pregnant woman in the same trimester who does not have HELLP syndrome). For example, if the preeclampsia biomarker is C5a or C5b-9, then the reference level of C5a or C5b-9 is obtained from the levels of C5a or C5b-9 of a healthy subject. In other embodiments, the reference level is the average reference level for the same preeclampsia biomarker from a population of healthy subjects. In other embodiments, the reference level is the average plus one or two standard deviations of the preeclampsia biomarker level for the same preeclampsia biomarker from a population of healthy subjects (e.g., reference range). In some embodiments, the population of healthy subjects can range from at least three healthy individuals to 25 healthy individuals, and even more than 50 healthy individuals.

In certain embodiments, the reference level is a normalized reference level. For example, for levels of biomarkers measured in urine, those levels can be normalized to creatinine.

### Biological Samples

Examples of biological samples include but are not limited to body fluids, whole blood, plasma, stool, intestinal fluids or aspirate, and stomach fluids or aspirate, serum, cerebral spinal fluid (CSF), urine, sweat, saliva, tears, pulmonary secretions, breast aspirate, prostate fluid, seminal fluid, cervical scraping, amniotic fluid, intraocular fluid, mucous, and moisture in breath. In particular embodiments of the method, the biological sample may be whole blood, blood plasma, blood serum, or urine. In certain embodiments, the biological sample is plasma. In certain embodiments, the biological sample is urine. When urine is used as the biological sample, the level of the preeclampsia biomarker can be normalized to urine creatinine levels or total protein levels.

### Non-Human Machines/Computer Implementation Systems and Methods

Various embodiments of the present invention provides for a non-transitory computer readable medium comprising instructions to execute the methods of the present invention, as described herein.

In certain embodiments, the methods of the invention implement a computer program for example, to compare the levels of preeclampsia biomarkers. For example, a non-transitory computer program can be used.

Numerous types of computer systems can be used to implement the analytic methods of this invention according to knowledge possessed by a skilled artisan in the bioinformatics and/or computer arts.

Several software components can be loaded into memory during operation of such a computer system. The software components can comprise both software components that are standard in the art and components that are special to the present invention. The methods of the invention can also be programmed or modeled in mathematical software packages that allow symbolic entry of equations and high-level specification of processing, including specific algorithms to be used, thereby freeing a user of the need to procedurally program individual equations and algorithms. Such packages include, e.g., Matlab from Mathworks (Natick, Mass.), Mathematica from Wolfram Research (Champaign, Ill.) or S-Plus from MathSoft (Seattle, Wash.). In certain embodiments, the computer comprises a database for storage of levels of preeclampsia biomarkers. Such stored profiles can be accessed and used to compare levels of preeclampsia biomarkers in the sample to known control/reference levels.

In addition to the exemplary program structures and computer systems described herein, other, alternative program structures and computer systems will be readily apparent to the skilled artisan. Such alternative systems, which do not depart from the above described computer system and programs structures either in spirit or in scope, are therefore intended to be comprehended within the accompanying claims.

Once a laboratory technician or laboratory professional or group of laboratory technicians or laboratory professionals determines the level of preeclampsia biomarkers, the same or a different laboratory technician or laboratory professional (or group) can analyze one or more assays to determine whether the level of preeclampsia biomarkers differs from the reference level or reference range, and then determine that the subject has preeclampsia or HELLP syndrome if the preeclampsia biomarker(s) do differ.

In various embodiments, provided herein is a non-transitory computer readable storage medium comprising: a storing data module containing data from a sample comprising a level of a preeclampsia biomarker; a detection module to detect the level of preeclampsia biomarker; a comparison module that compares the data stored on the storing data module with a reference data and/or control data, and to provide a comparison content, and an output module displaying the comparison content for the user, wherein an indication that the subject has preeclampsia or HELLP syndrome is displayed when the level of preeclampsia biomarkers differs from the reference level. In various embodiments, the reference level is a reference range.

In various embodiments, the control data comprises data from patients who do not have preeclampsia and/or HELLP syndrome.

Embodiments of the invention can be described through functional modules, which are defined by computer executable instructions recorded on a non-transitory computer readable media and which cause a computer to perform method steps when executed. The modules are segregated by function, for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The non-transitory computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can be accessed by a computer including and any suitable combination of the foregoing.

Computer-readable data embodied on one or more non-transitory computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine 2^{nd} ed. (CRC Press, London, 2005) and Ouelette and Bzevanis 3^{rd} Bioinformatics: A Practical Guide for Analysis of Gene and Proteins (Wiley & Sons, Inc., ed., 2004).

The functional modules of certain embodiments of the invention, include for example, a measuring module, a storage module, a comparison module, and an output module. The functional modules can be executed on one, or multiple, computers, or by using one, or multiple, computer networks. The measuring module has computer executable instructions to provide, e.g., expression information in computer readable form.

The measuring module, can comprise any system for detecting the levels of preeclampsia biomarkers.

The information determined in the determination system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon the level of preeclampsia biomarkers information. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising preeclampsia biomarker level information.

In one embodiment the reference data stored in the storage module to be read by the comparison module is, e.g., data from patients who do not have preeclampsia or HELLP syndrome.

The “comparison module” can use a variety of available software programs and formats for the comparison operative to compare binding data determined in the measuring module to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related, for example, preeclampsia biomarker levels.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content-based in part on the comparison result that may be stored and output as requested by a user using an output module.

The content based on the comparison result, may be preeclampsia biomarker levels compared to reference levels.

In various embodiments of the invention, the content based on the comparison result is displayed on a computer monitor. In various embodiments of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

### Treatment

Based on findings that preeclampsia is associated with complement activation, complement inhibitors can be used to treat preeclampsia or HELLP syndrome.

Various embodiments provide for a method of treating preeclampsia or HELLP syndrome, comprising administering a complement inhibitor to a subject in need thereof. In various embodiments, the subject in need thereof can be determined using the methods of the present invention. In other embodiments, the subject in need thereof can present with symptoms indicative of preeclampsia or HELLP syndrome.

In various embodiments, the preeclampsia therapy or HELLP syndrome therapy selected and/or administered as discussed herein are described below.

In various embodiments, the preeclampsia therapy or HELLP syndrome therapy comprises using a complement protein inhibitor. One example of a complement inhibitor is Eculizumab. Other examples include, but are not limited to Pexelizumab (short-acting C5 monoclonal Ab), Compstatin (C3 inhibitor), sCR1, and Heparin (unfractionated or low-molecular weight heparin).

Still other examples of complement inhibitor are provided in Tables 1-3, below. (See also, e.g., Makrides S C. Therapeutic inhibition of the complement system. PHARMACOL REV. 1998 March; 50(1):59-87.)

TABLE 1
Protein inhibitors

| | | Site/mode of | Disease | |
|---|---|---|---|---|
| Protein | Identity | action | indication/model | References |
| TP10 (sCR1) | Soluble CR1 | C3/C5 | ARDS (clinical); lung | Weisman et al, 1990a, b; |
| | | convertases; | allotransplantation | Dellinger et al., |
| | | classical/alternative | (clinical); | 1995, 1996; Levin et |
| | | | xenotransplantation | al., 1996; Marsh and |
| | | | | Ryan, 1997 |
| sCR1-SLe^{x} | sCR1 | C3/C5 | Pre-clinical | Picard et al, 1996; |
| | glycosylated | convertases; | | Bertino et al., 1996; |
| | with SLe^{x} | classical/alternative; | | Marsh and Ryan, 1997 |
| | | selectin- | | |
| | | mediated | | |
| sCR1[desLHR- | sCR1 minus | C3/C5 | In vitro | Scesney et al., 1996 |
| A] | LHR-A | convertases | Discordant | Gralinski et al., 1996 |
| | | alternative | xenotransplantation | |
| | | | Endothelial dysfunction | Lennon et al., 1996 |
| | | | Ischemia reperfusion | Murohara et al., 1995a |
| | | | injury | |
| sCR1[desLHR- | sCR1 minus | C3/C5 | In vitro | Marsh, 1997^{a} |
| A]-SLe^{x} | LHR-A | convertases; | | |
| | glycosylated | alternative; | | |
| | with SLe^{x} | selectin- | | |
| | | mediated | | |
| sCD59 | Soluble CD59 | MAC assembly | In vitro | Sugita et al, 1994; |
| | | | | Suzuki et al., 1996 |
| sDAF | Soluble DAF | C3/C5 | Reverse passive Arthus | Moran et al, 1992 |
| | | convertases | reaction | |
| | | Classical/alternative | In vitro | Christiansen et al., 1996 |
| sMCP | Soluble MCP | Factor I cofactor | Reverse passive Arthus | Christiansen et al., 1996 |
| | | activity | reaction | |
| Cl-INH | Cl esterase | Cl inactivation; | Reperfusion injury | Horstick et al., 1997; |
| | inhibitor | classical | | Buerke et al, 1995; |
| | | | | Murohara et al., 1995b |
| | | | Xenograft hyperacute | Dalmasso and Platt, |
| | | | rejection | 1993, 1994 |
| CAB-2 | Soluble chimeric | C3/C5 | Arthus reaction, | Higgins et al., 1997 |
| | MCP-DAF | convertases | Forssman shock | |
| | | Classical/alternative | In vitro | Iwata et al, 1994 |
| CD | Membrane-bound | C3/C5 | In vitro | Fodor et al, 1995 |
| | chimeric | convertases | | |
| | NH₂-CD59- | Classical/alternative | | |
| | DAF-GPI | | | |
| DC | Membrane-bound | C3/C5 | In vitro | Fodor et al., 1995 |
| | chimeric | convertases | | |
| | NH₂-DAF- | Classical/alternative | | |
| | CD59-GP1 | MAC assembly | | |
| N19-9 mAb | Anti-human C5 | C5, MAC | Cardiopulmonary | Würzner et al., 1991; |
| | murine | assembly | bypass | Rinder et al, 1995 |
| | monoclonal | | Ex vivo model | |
| | antibody | | | |
| N19-8 scFv | Anti-human C5 | C5, MAC | Myocardial reperfusion | Evans et al., 1995 |
| | murine single | assembly | injury | |
| | chain | | | |
| | Fv | | | |
| 5G1.1-SC | Anti-human C5 | C5, MAC | Cardiopulmonary | Thomas et al., 1996 |
| | humanized | assembly | bypass | |
| | scFv | | | |
| ClqR | 66-kDa ClqR, | Classical | In vitro | van den Berg et al., 1995 |
| | detergent- | | | |
| | solubilized | | | |
| C5aR | C5a oligopeptide | C5aR | In vitro; dermal | van Oostrum et al., 1996 |
| antagonists | analogs | | inflammation; | |
| | | | C5a-induced | |
| | | | neutropenia | |
| Factor J | Glycoprotein | Classical/alternative | In vitro | López-Trascasa et al., |
| | | | | 1989; González-Rubio |
| | | | | et al., 1994 |

^{a}Marsh H Combined complement inhibition and selectin binding by sCR1sLe^{x} and sCRI[deshlift-A]sLe^{x}.
Paper presented at “New Therapeutic Targets Based on Control of Complement System,” Jun. 9-11, 1997; Boston, MA.

TABLE 2
Synthetic inhibitors of complement activation

| | | | Structural | | | |
|---|---|---|---|---|---|---|
| Name | Compound Activity | MW | template/source | Site of action | IC₅₀ (μM) | References |
| | Peptide analogues | | C5a | C5aR | 2 | van Oostrum |
| | and derivatives | | | antagonist | | et al., 1996 |
| | Peptide | | C5a, C-terminal | C5aR | | Kawai et al., |
| | | | octapeptides | antagonist | | 1991; Kawai |
| | | | | | | et al., 1992 |
| | Peptide | | C5a, His⁶⁷- | C5aR | | Or et al., 1992 |
| | | | modified C- | antagonist | | |
| | | | terminal | | | |
| | | | octapeptide | | | |
| | | | analogues | | | |
| | | | C5a | C5aR | | Zhang et al., |
| | | | | antagonist | | 1997 |
| | Peptide | | C5a hexapeptide | C5aR | 0.070 | Konteatis et |
| | | | | antagonist | | al., 1994 |
| C089 | Peptide with | | | | | |
| | aromatic | | | | | |
| | substitutions | | | | | |
| PR226 | Peptide | 2157 | C5aR | C5aR: | | Baranyi et al., |
| | | | | antagonist at | | 1996 |
| | | | | >500 nM | | |
| | | | | agonist at | | |
| | | | | <2.0 nM | | |
| | Peptide | | C3a C-terminus | C3aR | | Kretzschmar et |
| | | | | antagonist | | al., 1992 |
| | Peptide | 1430 | C3b-based phage | C3 | Classical: | Sahu et al., |
| | | | display screening | | 63; | 1996 |
| | | | | | alternative: | |
| | | | | | 12 | |
| | Peptide | | CH₂ domain of | | | Boackle et al., |
| | | | human IgG | | | 1979; Lukas |
| | | | | | | et al., 1981 |
| | Peptide | | C1q | | | Reid et al., |
| | | | | | | 1977 |
| | Peptide containing | | | C3 convertase | | Kossorotow et |
| | Phe/Tyr | | | | | al., 1977 |
| | Peptide | | Factor B-related | Factor D, | | Lesavre et al., |
| | | | hexapeptides | alternative | | 1982 |
| CBP2 | Peptide | | C1q B chain helical | C1q | | Fryer et al., |
| | | | region | | | 1997 |
| | Peptide | | C3 | C3 | 25 | Ogata and |
| | | | | | | Low, 1997 |
| DFP | Diisopropyl | | | factor D | | Cole et al., |
| | fluorophosphates | | | | | 1997; Fearon |
| | (model | | | | | et al., 1974 |
| | compound) | | | | | |
| BCX- | | | | factor D | 0.096 | Kilpatrick, |
| 1470 | | | | | | 1997^{a} |
| | K-76 analogs | | K-76 (see table | | Classical: | Kaufman et |
| | | | below) | | 1,600; | al., 1995a,b |
| | | | | | alternative: | |
| | | | | | 2,500 | |
| TKIKc | K-76 derivative | | K-76 (see table | | Classical: | Sindelar et al., |
| | | | below) | | 160; | 1996 |
| | | | | | alternative: | |
| | | | | | 1,360 | |
| K-76 | K-76 derivative | | K-76 (see table | | C5 | Hong et al., |
| COOH | | | below) | | | 1979; |
| | | | | | | Miyazaki et |
| | | | | | | al., 1984; |
| | | | | | | Tanaka et |
| | | | | | | al., 1996 |
| FUT- | Nafamstat mesilate | | | Classical, | | Fujii and |
| 175 | | | | alternative | | Hitomi, |
| | | | | | | 1981; |
| | | | | | | Hitomi and |
| | | | | | | Fujii, 1982; |
| | | | | | | Ikari et al., |
| | | | | | | 1983; |
| | | | | | | Aoyama et |
| | | | | | | al., 1984; |
| | | | | | | Kreil et al., |
| | | | | | | 1989; |
| | | | | | | Issekutz et |
| | | | | | | al., 1990; |
| | | | | | | Homeister et |
| | | | | | | al., 1992; |
| | | | | | | Inose et al., |
| | | | | | | 1997 |
| PS- | Oligodeoxyribonucleotide | | | Classical, | | Shaw et al., |
| oligo | containing | | | alternative | | 1997 |
| | phosphorothioate | | | | | |
| | backbone | | | | | |
| | linkages | | | | | |
| | Other compounds | | | | | Fujii and |
| | | | | | | Aoyama, |
| | | | | | | 1984; |
| | | | | | | Asghar, |
| | | | | | | 1984 |

^{a}Kilpatrick, JM Development of small molecule inhibitors of factor D. Paper presented at: “New Therapeutic Targets Based on Control of the Complement System,” Jun. 9-11, 1997; Boston, MA.

TABLE 3
Naturally occurring compounds that inhibit complement activation

| | | | | Site of | IC₅₀ | |
|---|---|---|---|---|---|---|
| Compound | Identity | MW | Source | action/model | (μM) | References |
| Extract | Polyphenolic | | Plant pollen | | | Berrens et al., |
| | flavonoid | | | | | 1997 |
| Glycyrrhizin | β-glycyrrhetinic | | Glycyrrhiza | C2, classical, in | 35 | Kroes et al., |
| | acid steroid-like | | glabra roots | vitro | | 1997 |
| GR-2II | Polysaccharide | | Glycyrrhiza | In vitro | | Zhao et al., |
| | | | uralensis roots | | | 1991 |
| | Polysaccharide | 11,000 | Malva sylvestris | In vitro | | Gonda et al., |
| | | | leaves | | | 1990 |
| AGIIb-1, | Polysaccharide | | Angelica | In vitro | | Kiyohara et al., |
| AR-2IIa | | | acutiloba roots | | | 1989a,b; |
| | | | | | | Yamada et |
| | | | | | | al., 1987 |
| Rosmarinic | Ester | | Rosmarinus | C3 convertase, | 5-10 | Englberger et |
| acid | | | officinalis | classical, in | | al., 1988 |
| | | | | vitro | | |
| | | | Melissa | | | |
| | | | officinalis | | | |
| Extract | Polyanionic | | Ephedra sinica | C2, C9, | | Ling et al., |
| | carbohydrate | | | classical, | | 1995 |
| | | | | alternative, in | | |
| | | | | vitro | | |
| Extract | | | Eugenia | Classical | | Locher et al., |
| | | | malaccensis | inhibition, | | 1995 |
| | | | | alternative | | |
| | | | | activation, in | | |
| | | | | vitro | | |
| Extract | Alkaloid | | Berberis vulgaris | In vitro, in vivo | | Ivanovska and |
| | | | roots | (DTH | | Philipov, |
| | | | | reaction) | | 1996 |
| Extract | | | Fraxinus | | | Ivanovska et al., |
| | | | | | | 1996 |
| Extract | Polysaccharide | | Panax ginseng | In vitro | | Gao et al., 1989 |
| | | | roots, leaves | | | |
| BR-5-I | Polysaccharide | 18,500 | Bupleurum | In vitro | | Yamada et al., |
| | | | falcatum, roots | | | 1988 |
| Extract | | | Pokeweed | In vitro | | Gancevici and |
| | | | | | | Popescu, |
| | | | | | | 1987a; |
| | | | | | | Popescu et |
| | | | | | | al., 1988 |
| Extract | | | Spices | | | Gancevici and |
| | | | | | | Popescu, |
| | | | | | | 1987b |
| Extract | Proanthocyanidin | | Jatropha | Classical, in | | Kosasi et al., |
| | Ca⁺⁺-binding | | multifida latex | vitro | | 1989 |
| | polymer | | | | | |
| Fucan | Sulfated | 16,000-22,000 | Ascophyllum | C1, C4, factor | | Blondin et al., |
| | polysaccharide | | nodosum | B, classical, | | 1994; |
| | | | brown seaweed | alternative | | Charreau et |
| | | | | | | al., 1997 |
| CI | Glycoprotein | | Aspergillus | Alternative, in | | Washburn et al., |
| | | | fumigatus | vitro | | 1990 |
| Complestatin | Peptide-like | 1325 | Streptomyces | Classical, | | Kaneko et al., |
| | related to | | lavendulae | alternative, in | | 1980, 1989; |
| | glycopeptide | | | vitro, in vivo | | Seto et al., |
| | antibiotics | | | | | 1989; |
| | | | | | | Momota et |
| | | | | | | al., 1991 |
| K76 | Sesquiterpene | 402 | Stachybotrys | Classical, | | Miyazaki et al., |
| | | | complementi | alternative, in | | 1980; Kitano |
| | | | | vitro, in vivo | | et al., 1992; |
| | | | | | | Miyagawa et |
| | | | | | | al., 1993; |
| | | | | | | Kobayashi et |
| | | | | | | al., 1996 |
| Decorin | Dermatan sulfate | 100,000 | Extracellular | C1q, in vitro | | Krumdieck et |
| | proteoglycan | | matrix | | | al., 1992 |
| | | | (cartilage, | | | |
| | | | bone, skin, | | | |
| | | | cornea) | | | |
| Heparin | Glycosaminoglycan | | Mast cells, | C3 convertases, | | Weiler et al., |
| | | | basophils | classical, | | 1978, 1992; |
| | | | | alternative, in | | Linhardt et |
| | | | | vitro, in vivo | | al., 1988; |
| | | | | | | Maillet et al., |
| | | | | | | 1983, te |
| | | | | | | Velthuis et |
| | | | | | | al., 1996 |
| LU 51198 | Sulfated heparin | | | Myocardial | | Gralinski et al., |
| | fraction | | | injury, ex | | 1997 |
| | | | | vivo perfused | | |
| | | | | heart | | |
| Dextran | Glycosaminoglycan | 5000 | | Potentiates C1 | | Wuillemin et |
| sulfate | | | | inhibitor | | al., 1997 |
| GCRF^{a} | Chondroitin | | Glomerular | PC3bBb, factor | | Quigg, 1992 |
| | sulphate B | | epithelial cells | B? | | |
| | proteoglycan | | | | | |
| CSPG | Chondroitin | | B cell lines | C1q | | Kirschfink et |
| | sulphate | | | | | al., 1997 |
| | proteoglycan | | | | | |
| Extract | Phenolic | | Propolis (bee | C3, classical, | | Georgieva et |
| | | | product) | alternative, in | | al., 1997 |
| | | | | vitro | | |
| C4 | Serum protein | | Ginglymostoma | C4, in vitro, in | | Jensen, 1969 |
| inactivator | | | cirratum Nurse | vivo, | | |
| | | | shark | Forssman | | |
| | | | | shock, Arthus | | |
| | | | | reaction | | |
| L-156,602 | Cyclic | 822-839 | Streptomyces sp. | C5aR | | Hensens et al., |
| | hexadepsipeptide | | MA6348 | antagonist, in | | 1991; Tsuji et |
| | | | | vitro, in vivo | | al., 1992a,b |
| CVF^{b} | Glycoprotein | 144,000 | Cobra Naja naja | C3, in vitro, in | | Cochrane et al., |
| | | | | vivo | | 1970; |
| | | | | | | Schwartz and |
| | | | | | | Naff, 1971; |
| | | | | | | Müller |
| | | | | | | Eberhard and |
| | | | | | | Fjellström, |
| | | | | | | 1971; |
| | | | | | | Kourounakis |
| | | | | | | et al., 1973; |
| | | | | | | Jungi and |
| | | | | | | McGregor, |
| | | | | | | 1979 |
| M5 | Fibrinolytic | 25,000 | Crotalus | | | Chen and Rael, |
| | proteinase | | molossus | | | 1997 |
| | | | molossus | | | |

^{a}Glomerular complement regulatory factor.
^{b}Cobra venom factor; causes extensive complement activation, resulting in complement-depletion.

In other embodiments, the preeclampsia therapy or HELLP syndrome therapy comprises magnesium sulfate, an antihypertensive drug, or combinations thereof. In other embodiments, the preeclampsia therapy comprises corticosteroids to improve liver and platelet functioning to help prolong the pregnancy. In other embodiments, the preeclampsia therapy or HELLP syndrome therapy comprises anticonvulsive medications. In still other embodiments, the preeclampsia therapy comprises hydralazine, labetalol, nifedipine, sodium nitroprusside or a combination thereof.

In some embodiments, the preeclampsia therapy or HELLP syndrome therapy comprises fetal monitoring and tests including biophysical tests, sonograms, non-stress tests and fetal movement evaluation.

In some embodiments, the HELLP syndrome therapy comprises blood transfusion if platelet count gets too low.

### Formulation and Administration

In various embodiments, the preeclampsia therapy is delivered in a pharmaceutically acceptable carrier in a therapeutically effective amount.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration. Additional carrier agents, such as liposomes, can be added to the pharmaceutically acceptable carrier.

As used herein, the term “a therapeutically effective amount” refers an amount sufficient to achieve the intended purpose. For example, an effective amount of a preeclampsia therapy will cause a reduction or even completely halt one or more symptoms of preeclampsia, or prolong pregnancy. An effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, the terms “administering,” refers to the placement of a preeclampsia therapy into a subject by a method or route which results in at least partial localization of the preeclampsia therapy at one or more desired site(s). The preeclampsia therapy can be administered by any appropriate route which results in an effective treatment in the subject. “Route of administration” may refer to any administration pathway known in the art, including but not limited to aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

Therapeutic compositions contain a physiologically tolerable carrier together with an active agent as described herein, dissolved or dispersed therein as an active ingredient. In one embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. In one embodiment, the term “pharmaceutically acceptable” means approved by a regulatory agency of the Federal or a state government or listed in the U.S. Pharmacopeia or other generally recognized pharmacopeia for use in animals, and more particularly in humans.

The precise dose and formulation to be employed depends upon the potency of the agent, and include amounts large enough to produce the desired effect, e.g., a reduction in one or more symptoms of preeclampsia, or prolong pregnancy. The dosage should not be so large as to cause unacceptable adverse side effects. Generally, the dosage will vary with the type of preeclampsia therapy, and with the age, condition, and sex of the patient are also considered. Dosage and formulation of the preeclampsia therapy will also depend on the route of administration, and the seriousness and/or extent of the disease or disorder, and should be decided according to the judgment of the practitioner and each patient's circumstances. Effective doses can be extrapolated from dose-response curves derived from in vitro or animal model test systems.

The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 m/kg body weight. Alternatively, the dose range will be titrated to maintain serum levels between 5 μg/mL and 30 m/mL.

Administration of the doses recited above can be repeated for a limited period of time. In some embodiments, the doses are given once a day, or multiple times a day, for example but not limited to three times a day. In various embodiments, the doses recited above are administered daily for several weeks or months. The duration of treatment depends upon the subject's clinical progress and responsiveness to therapy. Continuous, relatively low maintenance doses are contemplated after an initial higher therapeutic dose.

Efficacy testing can be performed during the course of treatment using the methods described herein. Measurements of the degree of severity of a number of symptoms associated with a particular ailment are noted prior to the start of a treatment and then at later specific time period after the start of the treatment.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1A

### Methods

We enrolled singleton gestations ≧24 wks into a case-control study. Cases had severe preeclampsia or HELLP syndrome; controls were matched by gestational age and parity. Blood and urine were collected at study enrollment ELISA assays were utilized to measure C3a and C5a concentrations.

### Results

We evaluated 25 cases of severe preeclampsia/HELLP syndrome and 25 matched controls. Gestational age, parity, maternal age, and BMI were no different between groups. Plasma C3a and C5a levels were greater In cases vs. controls: median (IQ range) C3a 3549 ng/ml (2676-3869 ng/ml) vs. 2705 ng/ml (2460-2958 ng/ml), p 0.04; and C5a 33.5 ng/ml (29.8-42.0 ng/ml) vs. 23.6 ng/ml (18.9-27.8 ng/ml, p=0.0002. Urinary C3a was detectable in all subjects but not significantly different between groups (p=0.46); urinary C5a was detectable in only 16% of controls but 84% of cases (p<0.0001). Urinary measures of C5a were not correlated with total protein (p=0.70) or creatinine clearance (p=0.72).

### Conclusion

Complement dysregutation is common in severe preeclampsia/HELLP syndrome and targeted inhibition of the complement cascade represents a treatment strategy.

### Example 1B

### Methods

We performed a case-control study by prospectively enrolling 25 pregnant women with severe preeclampsia and matching them 1:1 to 25 healthy gravidas by gestational age (±2 wks) and parity (nulliparous or multiparous). Subjects were recruited from a cohort of women receiving care at Brigham and Women's Hospital from March through October 2012. Urine was collected on the day of enrollment, with complement activation determined by C3a, C5a and C5b-9 ELISA assays and proximal tubule injury by KIM-1 (kidney injury molecule 1) ELISA. Urinary protein concentrations were normalized to urine creatinine Comparisons between groups were analyzed using the ranksum test or spearman's correlation coefficient.

### Results

Subjects with severe preeclampsia were enrolled at mean(±SD) gestational age 32.3 (±4.2) wks, maternal age 30.6 (±5.2) yrs, BMI 31.8 (±6.0) kg/m 2, and 68% were nulliparous. Control subjects, matched by gestational age and parity, had slightly lower BMI (28.6±4.1 kg/m²). Severe preeclampsia was associated with increased proximal tubule injury compared to healthy controls, as measured by the ratio of urinary KIM-I (U-KIM1) to urine creatinine (U—Cr) [median ng/mg (U-KIM1/U—Cr) 1.60 vs. 0.62, p=0.0001]. Similar results were found when normalizing U-KIM1 levels to total urine protein. Increased urinary KIM-1 levels correlated strongly with increased levels of downstream complement proteins C5a (r=0.69, p<0.0001) and C5b-9 (r=0.59, p<0.0001) and these correlations remained strong even when limiting analysis to cases alone [C5a (r=0.60, p=0.001); C5b-9 (r=0.75, p<0.0001)]. In contrast, increased urinary KIM-1 levels in preeclampsia did not correlate with upstream complement protein C3a (r=0.17, p=0.43), due to exaggerated urinary excretion of KIM-1 relative to C3a. While levels of KIM-1, C5a and C5b-9 correlated with each other in preeclampsia they did not correlate with clinical markers of kidney injury including total urine protein, serum creatinine or uric acid, suggesting complement specific effects in the proximal tubule.

### Conclusion

Proximal tubule injury, as measured by urinary KIM-1, is increased in severe preeclampsia and correlates with complement activation. Downstream, rather than upstream, complement proteins correlate with kidney injury in severe preeclampsia indicating that terminal complement blockade has therapeutic rationale.

### Example 1C

### Methods

We prospectively enrolled 25 subjects with severe preeclampsia, 25 controls with chronic hypertension, 25 healthy controls without hypertension from a cohort of women receiving care at Brigham and Women's Hospital between March-October 2012. IRB approval was obtained through the Partners Human Research Committee. Pregnancies with multiple gestation or major fetal anomalies were excluded. Cases of severe preeclampsia, as defined by ACOG²³ (American Congress of Obstetricians and Gynecologists) (if ≧1 of the following criteria were present: (1) persistent blood pressure ≧160 mm Hg systolic or ≧110 mm Hg diastolic, (2) proteinuria ≧5 g in a 24-hour urine specimen, or ≧3+ in a random urine specimen, (3) oliguria <500 mL in 24 hours, (4) cerebral or visual disturbances, (5) pulmonary edema or cyanosis, (6) severe epigastric or right upper quadrant pain, (7) impaired liver function, defined by aspartate transaminase ≧70 U/L (normal range, 10-50 U/L), (8) thrombocytopenia <100K/μL (normal range, 150-450K/μL), or (9) fetal growth restriction <10th percentile for gestational age.) were recruited prospectively from the labor and delivery ward or antepartum units.

Blood pressure measurements were performed by licensed nurses or medical assistants, using automated digital manometers with the subject in sitting position and the arm at the level of the right atrium. Small, medium, or large blood pressure cuffs were used depending on body mass index (BMI). Control subjects had blood pressure measurements on the day of enrollment and additional measurements at subsequent antenatal visits to confirm proper group assignment. Eight subjects with chronic hypertension and 1 healthy control who developed preeclampsia after enrollment were excluded, being replaced by newly selected matched controls. Subjects with preeclampsia had serial blood pressures to confirm persistent blood pressure ≧140/90 mm Hg, and cases that were severe by blood pressure criteria had ≧2 measurements ≧160 mm Hg systolic or ≧110 mm Hg diastolic despite bed rest. All preeclampsia subjects had ≧300 mg protein on a 24-hour urine specimen or a spot urine protein/creatinine ratio ≧0.30 by clinical measurement. Controls with pre-existing chronic hypertension and healthy controls without pre-existing chronic hypertension or renal disease were recruited from prenatal clinics and matched 1:1 to cases with severe preeclampsia by parity (nulliparous or multiparous) and gestational age (±2 weeks). To minimize selection bias, every eligible case with severe preeclampsia identified during recruitment periods was approached for enrollment. Controls were identified from outpatient clinics as the first available subject meeting the matching criteria. Matching was confirmed by the treating provider who was blinded to demographic characteristics of the corresponding case. Healthy controls without chronic hypertension or renal disease were recruited from prenatal clinics and matched to cases by parity (nulliparous or multiparous) and gestational age (±2 wks).

Blood and urine samples were collected on the day of enrollment. Blood was collected in EDTA tubes and urine via a clean-catch specimen or Foley catheter. Samples were immediately centrifuged at −4° C., with supernatant aliquoted and stored at −80° C.

Complement activation was determined by human ELISA assays to measure C3a/C3a desArg (C3a), C5a/C5a desArg (C5a) and C5b-9 concentrations (BD Biosciences, San Jose, Calif.). Urinary placental growth factor (PlGF) levels were determined by human ELISA (R&D Systems, Minneapolis, Minn.). Samples were run in duplicate and a quality control sample (pooled plasma from cases) was utilized for determination of assay variability. Plasma samples were analyzed following a dilution of 10,000× (C3a) or 100× (C5a, C5b-9); urine samples were analyzed after an initial 2× dilution (C3a, C5a, and C5b-9) followed by 20-200× dilution for complement levels exceeding the top standard. Intra-assay coefficient of variation (CV) was 5.6% (C3a), 3.4% (C5a), and 4.5% (C5b-9); inter-assay CV was 11.1% (C3a), 13.8% (C5a) and 7.4% (C5b-9). Assay values within two standard deviations of the blank were considered below the lower limit of detection (LLD). The LLD for C3a ELISA was 0.013 ng/ml and for C5a and C5b-9 was 0.012 ng/ml.

Utilizing colorimetric methods, urine total protein was determined with the Pierce-BCA Protein Assay kit (Thermo Scientific, Rockford, USA) after separating interfering substances from the supernatant with the use of the Pierce Compat-Able Preparation Set (Thermo Scientific, Rockford, Ala.) and urine creatinine with the Parameter Creatinine Assay Kit (R&D Systems, Minneapolis, USA). Additional clinical variables (e.g., body mass index, blood pressure) and laboratory parameters (e.g., serum uric acid, 24-hr urine protein) were abstracted from medical records. The syndrome of hemolysis, elevated liver enzymes and low platelets (HELLP) was diagnosed by established criteria²⁴.

Before study initiation and based on previous studies, we estimated that 25 subjects per arm were required to demonstrate a 50% difference in plasma C3a, C5a, or C5b-9 levels between groups and a 2-fold difference in urine complement levels between groups, with α=0.05 and power=0.80. Plasma protein concentrations were presented as mean±SD, and urine protein concentrations were presented as medians (interquartile range). Significant differences in the levels of complement protein concentrations between groups were determined with the t-test (plasma) and ranksum test (urine). Correlations between complement levels and clinical parameters were determined utilizing Pearson's correlation coefficient (normally distributed variables) and Spearman's correlation coefficient (non-parametric variables). Data analysis was performed with Stata 10.0 (StataCorp, College Station, Tex.), and statistical significance was determined by a=0.05.

### Results:

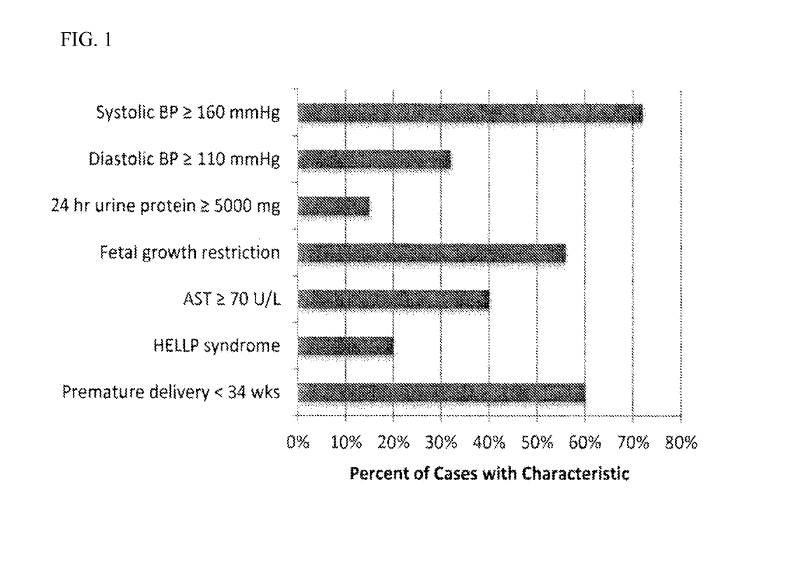

The baseline characteristics of the three study groups are provided in Table 4. Healthy nonhypertensive controls and hypertensive controls were matched to cases with severe preeclampsia by parity and gestational age at enrollment. Subjects with chronic hypertension or preeclampsia had higher BMI; chronic hypertensives were older; race was not significantly different between groups. Cases of severe preeclampsia were most commonly characterized by severe range blood pressures (72%), fetal growth restriction (56%), and transaminitis (40%); five cases (20%) had HELLP syndrome superimposed on severe preeclampsia and 15 (60%) required delivery before 34 weeks gestation. The peak systolic/diastolic blood pressure (mean±SD) among cases on day of enrollment was 176±20.7/104±11.0 mmHg, with 24 hour urine protein [median (range)] measuring 1020 mg (376-7329); 24 hr urine protein [median(IQ range)] measuring 1020 mg (682-3228). Descriptive clinical characteristics of cases are provided in FIG. 1.

TABLE 4
Characteristics of Study Subject By Group

| | | Chronic | Severe |
|---|---|---|---|
| | Healthy Control | Hypertension | Preeclampsia |
| | (n = 25) | (n = 25) | (n = 25) |
| Characteristics | | | |
| Gestational age at | 30.9 ± 4.8 | 31.3 ± 4.7 | 32.3 ± 4.2 |
| enrollment,* wk | | | |
| Gestational age at | 39.5 ± 1.2 | 38.2 ± 1.6 | 32.7 ± 4.0† |
| delivery, wk | | | |
| Peak SBP at | 111 ± 9.5 | 133 ± 12.1‡ | 176 ± 20.7† |
| enrollment, mm Hg | | | |
| Peak DBP at | 67.0 ± 7.9 | 82.3 ± 10.6‡ | 104 ± 11.0† |
| enrollment, mm Hg | | | |
| Urine total | 0.64 (0-1.3) | 0.64 (0.3-1.2) | 1.9 (1.6-4.0)∥ |
| protein/creatinine,§ | | | |
| mg/mg | | | |
| Nulliparous,* % | 68.0 | | |
| Maternal age, y | 29.0 ± 5.5 | 33.6 ± 5.9¶ | 30.6 ± 5.2 |
| Body mass index, | 28.6 ± 4.1 | 34.4 ± 9.2# | 31.8 ± 6.0** |
| kg/m² | | | |
| Race, % | | | |
| White | 44.0 | 52.0 | 60.0 |
| Black | 24.0 | 32.0 | 20.0 |
| Hispanic | 28.0 | 16.0 | 12.0 |
| Other | 4.0 | 0.0 | 8.0 |

Data are mean ± SD; comparisons between groups assessed by paired t tests and differences are nonsignificant (P > 0.05) unless otherwise specified.
CHTN indicates chronic hypertension; DBP, diastolic blood pressure; and SBP, systolic blood pressure.
*Controls matched 1:1 to cases by gestational age and parity.
†P < 0.0001, preeclampsia vs healthy controls or CHTN.
‡P < 0.0001, CHTN vs healthy controls.
§Data are medians (interquartile range).
∥Wilcoxon signed-rank test P < 0.01, preeclampsia vs healthy controls or CHTN.
¶P = 0.002, CHTN vs healthy controls.
#P = 0.003, CHTN vs healthy controls.
**P = 0.04, preeclampsia vs healthy controls.

Subjects with severe preeclampsia had higher levels of plasma C5a and C5b-9, but not C3a, compared to healthy controls; urinary levels of C5a and C5b-9 were even more exaggerated in preeclampsia (Table 5). Furthermore, urinary levels of C5a and C5b9 were detectable in 92% and 96% of cases, respectively, but only 52% and 8% of controls [OR 10.6, p=0.002 (C5a comparison); OR 276, p<0.0001 (C5b-9 comparison)]. Urinary C3a was detectable in all subjects and was modestly increased in preeclampsia.

TABLE 5
Plasma C3a, C5a, and C5b-9 in Cases and Controls

| | Healthy | Chronic | Severe |
|---|---|---|---|
| | Controls | Hypertension | Preeclampsia |
| Study Measure | (n = 25) | (n = 25) | (n = 25) |
| Plasma C3a | 3219 ± 548 | 3038 ± 656 | 3373 ± 916 |
| Plasma C5a | 31.6 ± 13.5 | 51.8 ± 9.9* | 40.8 ± 15.5† |
| Plasma C5b-9 | 348 ± 172 | 485 ± 233‡ | 444 ± 171‡ |

Data are mean ± SD (ng/mL); comparisons between groups assessed by paired t tests and differences are nonsignificant (P > 0.05), unless otherwise specified.
CHTN indicates chronic hypertension.
*P < 0.0001, CHTN vs healthy controls; P = 0.01, CHTN vs preeclampsia.
†P = 0.01, preeclampsia vs healthy controls.
‡P = 0.04, preeclampsia vs healthy controls; P = 0.02, CHTN vs healthy controls.

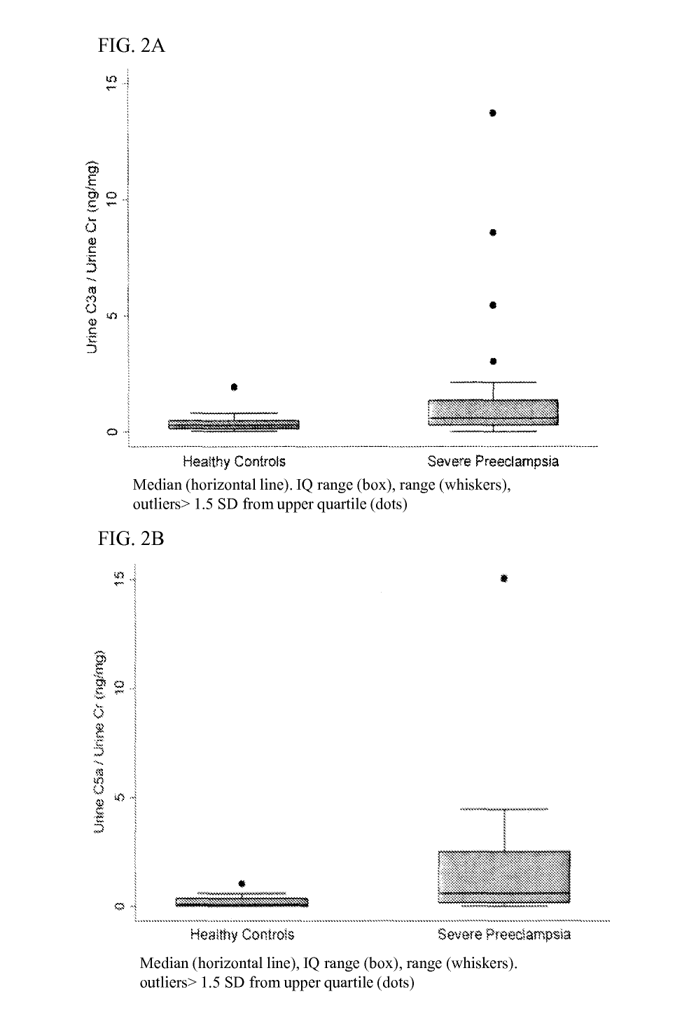

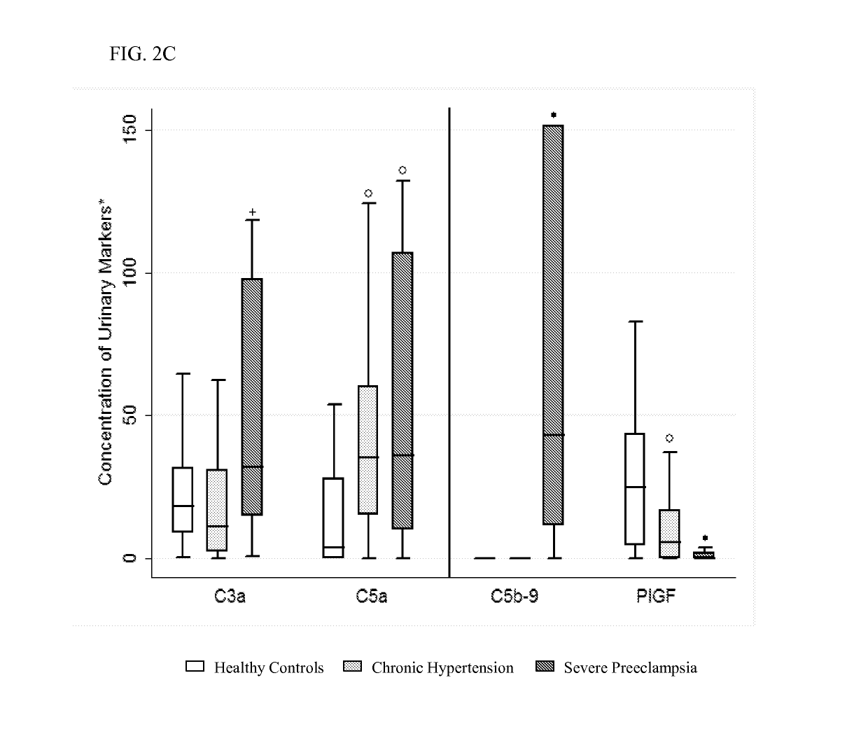

Preeclampsia cases had a greater ratio of urine total protein to creatinine when compared to controls [median (range) 12.7 (6.0-40.8) vs. 10.2 (6.7-15.7), p=0.01]. Given this discrepancy, and the knowledge that urinary protein excretion fluctuates throughout the day, we normalized urine complement levels to creatinine The ratios of urine C3a (UC3a) and C5a (U-05a) to urine creatinine (U—Cr) remained significantly greater in subjects with preeclampsia compared to controls [median ng/mg (range) U—C3a/U—Cr 9.22 (0.22-125) vs. 2.47 (0.29-12.9), p=0.008; U-05a/U—Cr 0.59 (0-15.1) vs. 0.11 (0-1.06), p=0.0005], (FIG. 2a-b). Similar results were found when normalizing urine C3a and C5a levels to total protein (data not shown). Comparisons of normalized urine C5b-9 measures were not displayed because only two controls had detectable levels of C5b9.

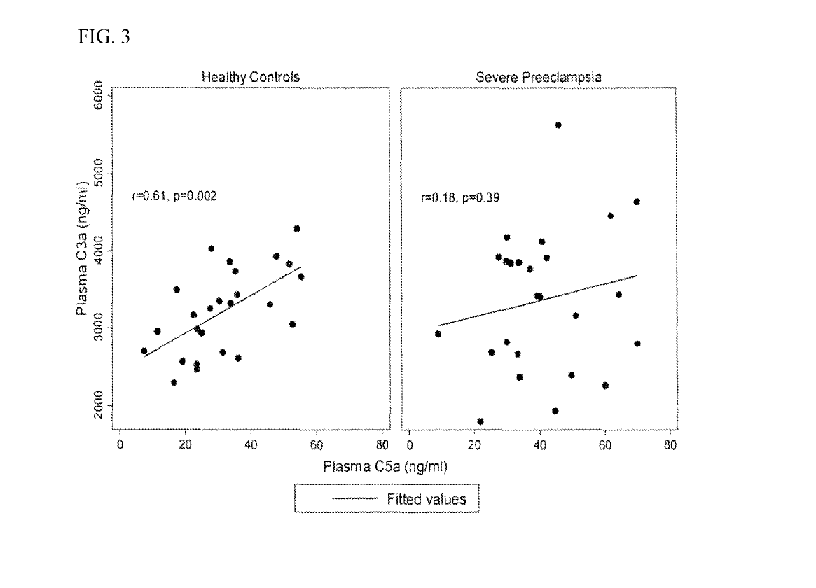

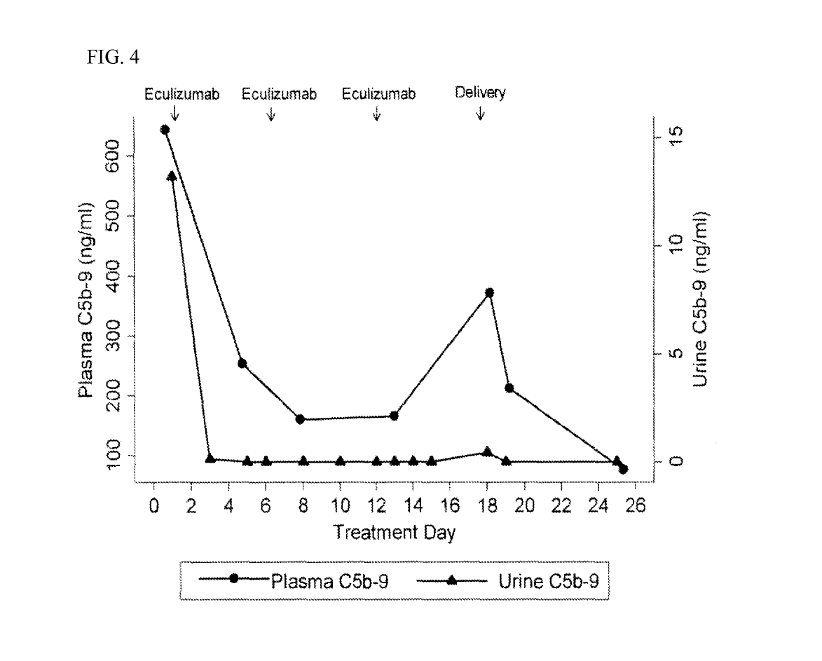

To evaluate dysregulation of complement signaling we evaluated correlations between plasma and urine complement levels in cases and controls. Interestingly, increased plasma levels of C3a correlated with increased plasma levels of C5a (r=0.61, p=0.002) in control subjects but not in those with severe preeclampsia (r=0.18, p=0.39), FIG. 3. Similarly, increased plasma levels of C5a correlated with increased urinary excretion of C5a in controls (r=0.46, p=0.02) but not cases (r=−0.03, p=0.89). Meanwhile, rising urine C5a levels in preeclampsia subjects were strongly associated with urinary C3a (r=0.76, p<0.0001) and C5b-9 (r=0.72, p=0.0001) but not levels of 24-hr total urine protein (r=0.29, p=0.21), serum creatinine (r=−0.27, p=0.15) or uric acid (r=0.03, p=0.87). Urine C5b-9 levels were also unrelated to serum creatinine and uric acid, but did correlate modestly with increased 24-hr urine protein measures (r=0.48, p=0.03).

Plasma levels of complement activation are displayed in Table 5. Subjects with severe preeclampsia or chronic hypertension had higher levels of plasma C5a and C5b-9, but not C3a, compared with healthy controls. Plasma levels of C3a, C5a, and C5b-9 did not correlate with peak systolic/diastolic blood pressure or BMI.

Urinary levels of complement activation are displayed in FIG. 2C. Urinary C3a levels were detectable in nearly all subjects but were significantly increased in severe preeclampsia group versus both control groups (P<0.01). These findings were unchanged after adjustment for U—Cr (median urine C3a/U—Cr [interquartile range], preeclampsia: 9.2 [2.0-20] ng/mg versus chronic hypertension: 1.7 [0.60-2.7] ng/mg and healthy controls: 2.5 [1.4-3.9] ng/mg; P<0.0001). Urine C3a correlated with total urine protein (r=0.30; P=0.01) and peak systolic blood pressure (r=0.24; P=0.04) but did not correlate with BMI.

Urinary C5a was detectable in 92% of subjects with severe preeclampsia or chronic hypertension compared with only 52% of healthy controls (P<0.001). C5a levels were not significantly different between preeclampsia and chronic hypertension groups but were greater in both these groups compared with healthy controls (FIG. 2C). These findings were unchanged after adjustment for U—Cr (median urine C5a/U—Cr [interquartile range], preeclampsia: 0.59 [0.17-2.5] ng/mg or chronic hypertension: 0.46 [0.27-0.81] ng/mg versus healthy controls: 0.11 [0-0.38] ng/mg; P<0.01). Urine C5a levels correlated with total urine protein (r=0.43; P=0.0001) and peak systolic blood pressure (r=0.36; P=0.002) but not BMI.

Urinary C5b-9 was detectable in 96% of subjects with severe preeclampsia compared with only 12% of controls with chronic hypertension and 8% of healthy controls (P<0.0001). Furthermore, urinary C5b-9 levels were markedly elevated in severe preeclampsia group compared with both control groups (FIG. 2C). Adjustment for U—Cr was not performed because few controls had detectable C5b-9 levels. Urine C5b-9 levels correlated with total urine protein (r=0.34; P=0.003) and peak systolic blood pressure (r=0.74; P<0.0001) but not BMI.

To validate an altered angiogenic state in our subjects, we investigated urinary PlGF levels (FIG. 2C). Urinary PlGF was detectable in only 28% of subjects with severe preeclampsia but in 76% of both controls with hypertension and healthy controls (P<0.001). PlGF levels were significantly lower in the preeclampsia group compared with the group with chronic hypertension, and levels in both these groups were significantly lower than those in healthy controls. Similar results were obtained after adjustment for U—Cr (median urine PlGF/U—Cr [interquartile range], preeclampsia: 0 [0-18] μg/mg versus chronic hypertension: 59 [11-160] pg/mg and healthy controls: 238 [70-549] pg/mg; P<0.0001). Urinary PlGF levels showed a strong inverse correlation with urinary C5b-9 levels (r=−0.41; P=0.003) and peak systolic blood pressure (r=−0.57; P<0.0001) but did not correlate with C3a, C5a, total urine protein, or BMI.

Finally, we evaluated correlations among cases with preeclampsia only. Within the group of cases with severe preeclampsia, there were strong positive correlations between urinary levels of C3a, C5a, and C5b-9 (C3a and C5a [r=0.76; P<0.0001], C3a and C5b-9 [r=0.59; P=0.002], and C5a and C5b-9 [r=0.71; P=0.0001]). However, urine levels of C3a, C5a, and C5b-9 did not correlate with plasma complement levels, serum creatinine, or uric acid.

### Example 2

A 35 year old nulliparous female at 26 weeks gestation, with no significant medical history and normal BMI (19.8 kg/m²) presented to her local hospital with severe epigastric pain. On exam, she was afebrile with normal blood pressure (105/71 mmHg); she had a mild transaminitis (AST 61 and ALT 128 U/L), but abdominal ultrasound and magnetic resonance cholangiopancreatography were normal. She was discharged home with precautions, returning two days later with hypertension (159/108 mmHg) and labwork revealing elevated LDH (415 U/L. normal 135-225 U/L), decreased haptoglobin (25 mg/dL, normal 36-195 mg/dL), elevated liver enzymes (AST 494 and ALT 460 U/L. normal 10-50 U/L), and low platelets (84 K/uL, normal 150-450 K/uL). She was diagnosed with HELLP syndrome; betamethasone was administered for fetal maturation and magnesium sulfate for seizure prophylaxis, and she was transferred to our institution for anticipated delivery and neonatal support.

On arrival the patient was afebrile with normal neurologic status; blood pressure was 161/88 mmHg and labwork confirmed ongoing hemolysis, elevated liver enzymes and low platelets. Since there are several imitators of severe preeclampsia/HELLP syndrome [7], we performed an extensive work-up for alternative etiologies; also, review of medical history and prenatal records discovered no pre-existing hypertension, renal disease, or clotting disorder.

Routine studies, including serum creatinine, bilirubin, fibrinogen, coagulation studies, and glucose were normal; ANA screen and viral hepatitis panel were negative. Given no evidence for acute fatty liver of pregnancy, thrombotic thrombocytopenic purpura, hemolytic uremic syndrome, or systemic lupus erythematosus, we felt that the clinical presentation was most consistent with severe preeclampsia/HELLP syndrome. Ultrasound estimated fetal weight was 893 g (11th percentile) with normal amniotic fluid and diminished umbilical artery dopplers. Neonatal consult was obtained, after which the patient expressed reticence to be delivered due to newborn risks of marked prematurity and inquired about alternative treatment approaches. We discussed preeclampsia/HELLP syndrome as a systemic inflammatory disorder with the complement cascade a key mediator. Eculizumab, an FDA-approved C5 inhibitor, was offered as a putative treatment to prolong pregnancy; the patient agreed.

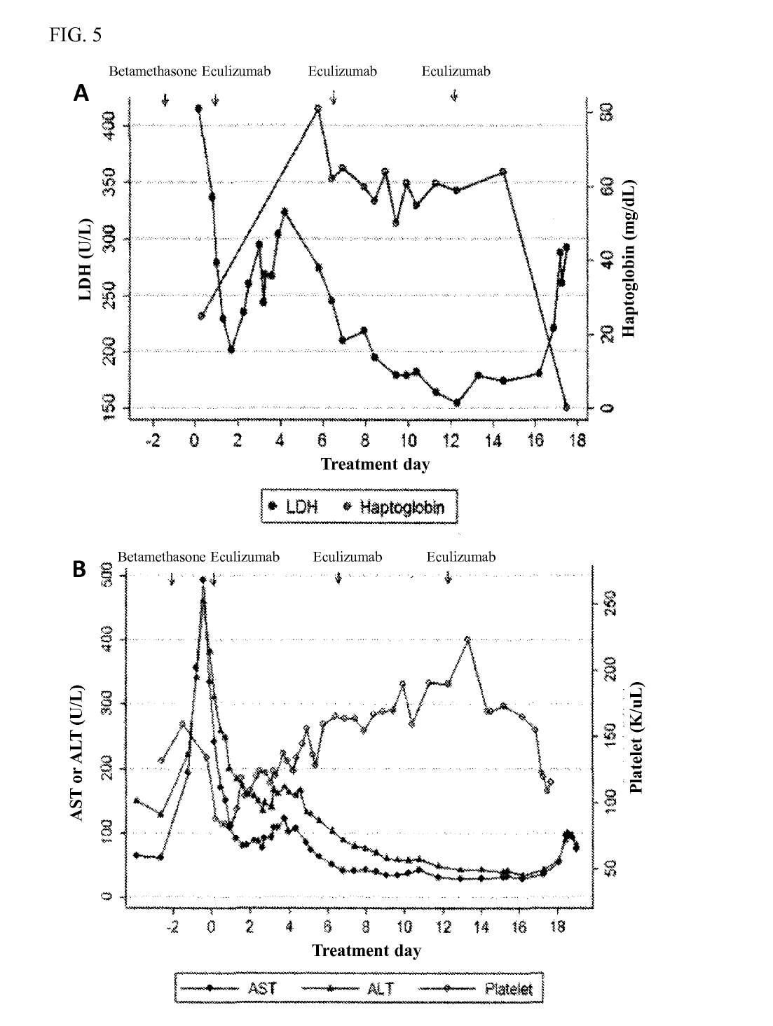

After informed consent and meningococcal vaccination (due to the small risk of Neisseria meningitidis with complement inhibition) treatment with Eculizumab (1200 mg) was commenced. The patient's initial course was complicated by escalating blood pressures and pulmonary edema, which responded to Labetalol and Lasix, respectively. The pulmonary edema, thought secondary to a combination of preeclamptic capillary leak and iatrogenic volume overload, did not recur after Lasix treatment. By treatment day 6, LDH, haptoglobin, AST, and platelet count normalized, FIG. 5a-b.

Fetal status was reassuring as indicated by continuous fetal heart monitoring, daily biophysical profile and umbilical artery dopplers; a 24 h urine collection was completed and measured 383 mg protein. Utilizing established regimens for treating PNH (paroxysmal nocturnal hemoglobinuria) and aHUS (atypical hemolytic uremic syndrome) Eculizumab was re-dosed on treatment day 7. That day blood pressures rose necessitating addition of Nifedipine. Of note, a trough level of Eculizumab was sub-therapeutic to block complement-dependent hemolysis (Alexion Pharmaceuticals). By 24 h after the second infusion, blood pressures improved and Labetalol was discontinued. In the setting of maternal and neonatal stability, and normal lab parameters, a third dose of Eculizumab was given on treatment day 13, one day early due to the prior subtherapeutic trough on day 7.

On treatment day 16, a 24 h urine collection measured 4522 mg of protein. Concomitantly, blood pressures rose (140-160/90-100 mmHg) and laboratory parameters began to worsen, FIG. 5a-b. Due to worsening status, delivery was recommended. Cesarean delivery was performed at 29 2/7 wks gestation, resulting in a male neonate weighing 1220 g, with Apgars 4 and 8 at 1 and 5 min. The placenta was severely fragmented, weighing 155 g. Post-operatively there was spontaneous diuresis; blood pressure and lab parameters improved. Two units of packed red blood cells were transfused for symptomatic anemia (Hgb 6.9 g/dL). The patient was discharged on post-operative day 4 and remained well postpartum, discontinuing Nifedipine at 6 weeks. Postpartum evaluation for antiphospholipid antibodies was negative.

The neonate was intubated at 10 min of life for respiratory support and admitted to the NICU. He was extubated the following morning after one dose of surfactant; blood cultures were negative; and head ultrasound was normal. NICU course was notable for a patent ductus arteriosus (responsive to Indomethacin) and inguinal hernia repair (day 67), after which he was discharged home in good condition. There were trace levels of Eculizumab in cord blood samples taken at delivery, thought secondary to contamination with maternal blood. The cord blood level of Eculizumab (15 ug/ml) was too low to block complement and was 20-fold lower than the maternal concentration; there was no detectable Eculizumab in the breast milk (Alexion Pharmaceuticals).

In preeclampsia, excessive aponecrotic feto-placental debris in the maternal circulation[8] leads to systemic inflammation[4], angiogenic dysregulation[9], altered eicosanoid metabolism, elevated lipid peroxides, and oxidative stress, culminating in clinical disease[6]. The complement system is a key mediator of systemic inflammation and is activated in preeclampsia and HELLP syndrome [5,10,11]. Complement activation induces dysregulation of angiogenic factors, therefore demonstrating a proximal role for complement in adverse pregnancy outcomes [12] Inhibition of C5 results in a decrease in sFLT-1 and an increase in VEGF [12] and reduced formation of the terminal complement complex C5b-9 which localizes to sites of villous trophoblast injury in preeclampsia [13]. Eculizumab, a monoclonal antibody inhibitor of C5, reduces generation of complement components C5a and C5b-9 and their downstream effects [14]. It is FDA-approved for PNH and aHUS, appears safe to use in pregnancy [15], and is compelling as a novel therapeutic strategy for preeclampsia. While there is potential for unrecognized fetal risks, Eculizumab lacks the Fe region necessary for transport of IgG compounds across the placenta, therefore likely minimizing fetal exposure [14,15].

Clinical studies evaluating expectant management of severe preeclampsia/HELLP syndrome have reported complete biochemical resolution in 29-43% of subjects; however, fetal and/or neonatal death rates were high (14-20%) [2,3]. Furthermore, due to the lack of maternal benefit and the unacceptably high fetal loss rates, expectant management of HELLP syndrome greater than 48 h, even in the setting of betamethasone administration, is discouraged [1,16].

We decided to offer Eculizumab as salvage therapy for severe preeclampsia/HELLP syndrome precisely because bed rest alone and betamethasone do not appear sufficient. We believe this is the first use of Eculizumab for the treatment of preeclampsia and HELLP syndrome, which resulted in marked improvement in clinical and laboratory parameters without apparent adverse effects. While not wishing to be bound by any particular theory, we believe in our case that increasing placental mass and feto-placental debris ultimately tipped the balance toward inflammation, overwhelming the initial complement inhibition and resulting in resurgent disease. Future investigations would help correlate the clinical changes seen with levels of investigational biomarkers such as complement proteins, angiogenic markers or inflammatory cytokines Nevertheless, pregnancy was prolonged by 17 days, allowing further fetal growth and maturation and likely resulting in reduced neonatal morbidity with its associated short and long-term health care costs.

The use of Eculizumab as described herein supports a benefit of C5 inhibition for the treatment of severe preeclampsia/HELLP syndrome. Its use may be particularly helpful among women with mutations in complement regulatory proteins, which are found in 8-18% of women with severe preeclampsia [17].

Publications in Example 2
[1] Publications Committee. Society for Maternal-Fetal Medicine, Sibai B M. Evaluation and management of severe preeclampsia before 34 weeks' gestation. Amj Obstet Gynecol 2011; 205:191-8.
[2] Visser W, Wallengburg H C. Temporising management of severe pre-eclampsia with and without the HELLP syndrome. Br J Obstet Gynaeco/1995; 102:111-7.
[3] van Pampus M G. Wolf H. Westenberg S M. van der Post J A. Honsel G J, Treffers P E. Maternal and perinatal outcome after expectant management of the HELLP syndrome compared with pre-eclampsia without HELLP syndrome. Eur J Obstet Gynecol Reprod Bioi 1998; 76:31-6.
[4] Redman c w. Tannetta D S. Dragovic R A. Gardiner C, Southcombe J H, Collett G P, et al. Review: does size matter? Placental debris and the pathophysiology of pre-eclampsia. Placenta 2012; 33:548-54.
[5] Haeger M, Unander M, Norder-Hansson B, Tylman M, Bengtsson A. Complement, neutrophil and macrophage activation in women with severe preeclampsia and syndrome of hemolysis, elevated liver enzymes, and low platelet count. Obstet Gynecol 1992; 79:19-26.
[6] Feinberg B B. Preeclampsia: the death of Goliath. Am J Reprod Immunol 2006; 55:84-98.
[7] Sibai B M. Imitators of severe preeclampsia. Obstet Gyneco/2007; 109:956-66.
[8] Huppertz B. Kingdom J, Caniggia I. Desoye G, Black S. Korr H, et al. Hypoxia favors necrotic versus apoptotic shedding of placental syncytiotrophoblast into the maternal circulation. Placenta 2003; 24:181-90.
[9] Levine R J, Maynard S E, Qian C, Lim K H, England L J, Yu K F, et al. Circulating angiogenic factors and the risk of preeclampsia. N Engl J Med 2004; 350: 672-83.
[10] Derzsy z. Prohaszka z. Riga J, Fust G, Molvarec A. Activation of the complement system in normal pregnancy and preeclampsia. Mol Immunol 2010; 47: 1500-6.
[11] de Messias-Reason I J, Aleixo V, de Freitas H. Nisihara R M. Mocelin V, Urbanetz A. Complement activation in Brazilian patients with preeclampsia. J Investig Allergol Clin Immunol 2000; I 0:209-14.
[12] Girardi G. Yarilin D. Thurman J M, Holers V M, Salmon J E. Complement activation induces dysregulation of angiogenic factors and causes fetal rejection and growth restriction. J Exp Med 2006; 203:2165-75.
[13] Rampersad R, Barton A. Sadovsky Y, Nelson D M. The C5b-9 membrane attack complex of complement activation localizes to villous trophoblast injury in vivo and modulates human trophoblast function in vitro. Placenta 2008; 29: 855-61.
[14] Thomas T C. Rollins S A. Rother R P. Giannoni M A, Hartman S L. Elliot E A. et al. Inhibition of complement activity by humanized anti-CS antibody and single chain Fv. Mo. Immunol 1996; 33: 1389-401.
[15] Kelly R. Arnold L, Richards S, Hill A. Bomken C, Hanley J, et al. The management of pregnancy in paroxysmal nocturnal hemoglobinuria on long term eculizumab. Br J Haematol 2010; 149:446-50.
[16] Martin Jr J N, Rose C H, Briery C M. Understanding and managing HELLP syndrome: the integral role of aggressive glucocorticoids for mother and child. Amj Obstet Gynecol 2006; 195:914-34.
[17] Salmon J E, Heuser C. Triebwasser M, Liszewski M K. Kavanagh D, Roumenina L, et al. Mutations in complement regulatory proteins predispose to preeclampsia: a genetic analysis of the PROMISSE cohort. PLoS Med 2011; 8:e1001013.

### REFERENCES

### Superscript

1. Richani K, Soto E, Romero R, Espinoza J, Chaiworapongsa T, Nien J K, et al. Normal pregnancy is characterized by systemic activation of the complement system. J Matern Fetal Neonatal Med. 2005; 17:239-45.
2. Smith S C, Baker P N, Symonds E M. Placental apoptosis in normal human pregnancy. Am J Obstet Gynecol. 1997; 177:57-65
3. Lo Y M, Corbetta N, Chamberlain P F, Rai V, Sargent I L, Redman C W, et al. Presence of fetal DNA in maternal plasma and serum. Lancet. 1997; 350:485-7.
4. Masson P L, Delire M, Cambiaso C L. Circulating immune complexes in normal human pregnancy. Nature. 1977; 266:542-3.
5. Lau T W, Leung T N, Chan L Y, Lau T K, Chan K C, Tam W H, et al. Fetal DNA clearance from maternal plasma is impaired in preeclampsia. Clin Chem. 2002; 48:2141-6
6. Leung T N, Zhang J, Lau T K, Chan L Y, Lo Y M. Increased maternal plasma fetal DNA concentrations in women who eventually develop preeclampsia. Clin Chem. 2001; 47:137-9.
7. Huppertz B, Kingdom J, Caniggia I, et al. Hypoxia favors necrotic versus apoptotic shedding of placental syncytiotrophoblast into the maternal circulation. Placenta 2003; 24:181-90.
8. Feinberg B B. Preeclampsia: The death of Goliath. Am J Repro Immunol. 2006; 55:84-98.
9. Redman C W, Tannetta D S, Dragovic R A, et al. Review: Does size matter? Placental debris and the pathophysiology of pre-eclampsia. Placenta 20 12; 33:S48-54
10. Haeger M, Unander M, Norder-Hansson B, Tylman M, Bengtsson A. Complement, neutrophil, and macrophage activation in women with severe preeclampsia and syndrome of hemolysis, elevated liver enzymes, and low platelet count. Obstet Gynecol 1992; 79:19-26.
11. de Messias-Reason I J, Aleixo V, de Freitas H, Nisihara R M, Mocelin V, Urbanetz A. Complement activation in Brazilian patients with preeclampsia. J Investig Allergol Clin Immunol 2000; 10:209-14
12. Derzsy Z, Prohaszka Z, Rigo J, Fust G, Molvarec A. Activation of the complement system in normal pregnancy and preeclampsia. Mol Immunol 2010; 47:1500-06
13. Buurma A, Cohen D, Veraar K, Schonkeren D, Claas F H, Bruijn J A, et al. Preeclampsia is characterized by placental complement dysregulation. Hypertension. 2012; 60:1332-7.
14. Sakuma M, Morooka T, Wang Y, Shi C, Croce K, Gao H, et al. The intrinsic complement regulator decay-accelerating factor modulates the biological response to vascular injury. Arterioscler Thromb Vase Bioi. 2010; 30:1196-1202.
15. Kerr H, Richards A. Complement-mediated injury and protection of endothelium: Lessons from atypical haemolytic uraemic syndrome. Immunobiology, 20 12; 217:195-203
16. Girardi G, Yarilin D, Thurman J M, Holers V M, Salmon J E. Complement activation induces dysregulation of angiogenic factors and causes fetal rejection and growth restriction. J Exp Med 2006; 203:2165-75
17. Lynch A M, Salmon J E. Dysregulated complement activation as a common pathway of injury in preeclampsia and other pregnancy complications. Placenta. 2010; 31:561-7.
18. Haeger M, Unander M, Andersson B, Tarkowski A, Arnestad J P, Bengtsson A. Increased release of tumor necrosis factor alpha and interleukin-6 in women with the syndrome of hemolysis, elevated liver enzymes, and low platelet count. Acta Obstet Gynecol Scand. 1996. 75:695-701.
19. Redman C W, Sacks G P, Sargent I L. Preeclampsia: an excessive maternal inflammatory response to pregnancy. Am J Obstet Gynecol. 1999; 180:499-506.
20. Peng Q, Li K, Smyth L A, Xing G, Wang N, Meader L. C3a and C5a promote renal ischemia-reperfusion injury. J Am Soc Nephrol. 2012; 23:1474-85
21. Zhou W, Farrar C A, Abe K, Pratt J R, Marsh J E, Wang Y, et al. Predominant role for C5b-9 in renal ischemia/reperfusion injury. J Clin Invest. 2000; 105:1363-71
22. ACOG committee on practice bulletins, Obstetrics. ACOG Practice Bulletin. Diagnosis and management of preeclampsia and eclampsia. Obstet Gynecol. 2002; 99:159-67
23. Sibai B M. HELLP syndrome (hemolysis, elevated liver enzymes, and low platelets): much ado about nothing? Am J Obstet Gynecol. 1990; 162:311-6.
24. Guo R F, Ward P A. Role ofC5a in inflammatory responses. Annu Rev Immunol. 2005; 23:821-52.
25. M arder S R, Chenoweth D E, Goldstein I M, Perez H D. Chemotactic responses of human peripheral blood monocytes to the complement-derived peptides C5a and C5a desArg. J Immunol. 1985; 134:3325-
26. Nauta A J, Daha M R, Tijsma 0, van de Water B, Tedesco F, Roos A. The membrane attack complex of complement induces caspase activation and apoptosis. Eur J Immunol. 2002; 32:783-92.
27. Sims P J, Faioni E M, Wiedmer T, Shattil S J. Complement proteins C5b-9 cause release of membrane vesicles from the platelet surface that are enriched in the membrane receptor for coagulation factor Va and express prothrombinase activity. J Biol Chem. 1988; 263:18205-18212.
28. Oikonomopoulou K, Ricklin D, Ward P A, Lambris J D. Interactions between coagulation and complement—their role in inflammation. Semin Immunopathol. 20 12; 34:151-65.
29. Rampersad R, Barton A, Sadovsky Y, Nelson O M. The C5b-9 membrane attack complex of complement activation localizes to villous trophoblast injury in vivo and modulates human trophoblast function in vitro. Placenta 2008; 29:855-61.
30. Oppermann M, Gotze O. Plasma clearance of the human C5a anaphylatoxin by binding to leucocyte C5a receptors. Immunology. 1994; 82:516-21
31. Amara U, Flierl M A, Rittirsch D, Klos A, Chen H, Acker B, et al. Molecular intercommunication between the complement and coagulation systems. J Immunol. 2010; 185:5628-36.
32. Huber-Lang M, Sarma J V, Zetoune F S, Rittirsch D, Neff T A, McGuire S R. et al. Generation of C5a in the absence of C3: a new complement activation pathway. Nat Med. 2006; 12:682-7.
33. Salmon J E, Heuser C, Triebwasser M, Liszewski M K, Kavanagh D, Roumenina L, et al. Mutations in complement regulatory proteins predispose to preeclampsia: a genetic analysis of the PROMISSE cohort. PLoS Med. 2011; 8:e1001013.
34. Burwick R M, Feinberg B B. Eculizumab for the treatment of severe preeclampsia/HELLP syndrome. Placenta. 2012 (in-press).

### REFERENCES

### Parenthesis

1. Richani K, Soto E, Romero R, Espinoza J, Chaiworapongsa T, Nien J K, Edwin S, Kim Y M, Hong J S, Mazor M. Normal pregnancy is characterized by systemic activation of the complement system. J Matern Fetal Neonatal Med. 2005; 17:239-245.
2. Smith S C, Baker P N, Symonds E M. Placental apoptosis in normal human pregnancy. Am J Obstet Gynecol. 1997; 177:57-65
3. Lo Y M, Corbetta N, Chamberlain P F, Rai V, Sargent I L, Redman C W, Wainscoat J S. Presence of fetal DNA in maternal plasma and serum. Lancet. 1997; 350:485-487.
4. Masson P L, Delire M, Cambiaso C L. Circulating immune complexes in normal human pregnancy. Nature. 1977; 266:542-543.
5. Buurma A, Cohen D, Veraar K, Schonkeren D, Claas F H, Bruijn J A, Bloemenkamp K W, Baelde H J. Preeclampsia is characterized by placental complement dysregulation. Hypertension. 2012; 60:1332-1337.
6. Huppertz B, Kingdom J, Caniggia I, Desoye G, Black S, Korr H, Kaufmann P. Hypoxia favors necrotic versus apoptotic shedding of placental syncytiotrophoblast into the maternal circulation. Placenta. 2003; 24:181-190.
7. Lau T W, Leung T N, Chan L Y, Lau T K, Chan K C, Tam W H, Lo Y M. Fetal DNA clearance from maternal plasma is impaired in preeclampsia. Clin Chem. 2002; 48:2141-2146
8. Leung T N, Zhang J, Lau T K, Chan L Y, Lo Y M. Increased maternal plasma fetal DNA concentrations in women who eventually develop preeclampsia. Clin Chem. 2001; 47:137-139.
9. Feinberg B B. Preeclampsia: The death of Goliath. Am J Repro Immunol. 2006; 55:84-98.
10. Redman C W, Tannetta D S, Dragovic R A, Gardiner C, Southcombe J H, Collett G P, Sargent I L. Review: Does size matter? Placental debris and the pathophysiology of pre-eclampsia. Placenta. 2012; 33:S48-54.
11. Lynch A M, Gibbs R S, Murphy J R, Giclas P C, Salmon J E, Holers V M. Early elevations of the complement activation fragment C3a and adverse pregnancy outcomes. Obstet Gynecol. 2010; 117:75-83.
12. Lynch A M, Murphy J R, Byers T, Gibbs R S, Neville M C, Giclas P C, Salmon J E, Holers V M. Alternative complement pathway activation fragment Bb in early pregnancy as a predictor of preeclampsia. Am J Obstet Gynecol. 2008; 198:385.e1-9.
13. Haeger M, Unander M, Norder-Hansson B, Tylman M, Bengtsson A. Complement, neutrophil, and macrophage activation in women with severe preeclampsia and syndrome of hemolysis, elevated liver enzymes, and low platelet count. Obstet Gynecol. 1992; 79:19-26.
14. de Messias-Reason I J, Aleixo V, de Freitas H, Nisihara R M, Mocelin V, Urbanetz A. Complement activation in Brazilian patients with preeclampsia. J Investig Allergol Clin Immunol. 2000; 10:209-214.
15. Derzsy Z, Prohaszka Z, Rigo J, Fust G, Molvarec A. Activation of the complement system in normal pregnancy and preeclampsia. Mol Immunol. 2010; 47:1500-1506.
16. Denny K J, Coulthard L G, Finnell R H, Callaway L K, Taylor S M, Woodruff T M. Elevated complement factor C5a in maternal and umbilical cord plasma in preeclampsia. J Reprod Immunol. 2013; 97:211-216.
17. Peng Q, Li K, Smyth L A, Xing G, Wang N, Meader L. C3a and C5a promote renal ischemia-reperfusion injury. J Am Soc Nephrol. 2012; 23:1474-1485.
18. Zhou W, Farrar C A, Abe K, Pratt J R, Marsh J E, Wang Y, Stahl G L, Sacks S H. Predominant role for C5b-9 in renal ischemia/reperfusion injury. J Clin Invest. 2000; 105:1363-1371.
19. Lehto T, Honkanen E, Teppo A M, Meri S. Urinary excretion of protectin (CD59), complement S C5b-9 and cytokines in membranous glomerulonephritis. Kidney Int. 1995; 47: 1403-1411.
20. Kon S P, Coupes B, Short C D, Solomon L R, Raftery M J, Mallick N P, Brenchley P E. Urinary C5b-9 excretion and clinical course in idiopathic human membranous nephropathy. Kidney Int. 1995; 48:1953-1958.
21. Muller T F, Kraus M, Neumann C, Lange H. Detection of renal allograft rejection by complement components C5a and TCC in plasma and urine. J Lab Clin Med. 1997; 129:62-71.
22. ACOG committee on practice bulletins, Obstetrics. ACOG Practice Bulletin. Diagnosis and management of preeclampsia and eclampsia. Obstet Gynecol. 2002; 99:159-167.
23. Sibai B M. HELLP syndrome (hemolysis, elevated liver enzymes, and low platelets): much ado about nothing? Am J Obstet Gynecol. 1990; 162:311-316.
24. Haeger M, Unander M, Andersson B, Tarkowski A, Arnestad J P, Bengtsson A. Increased release of tumor necrosis factor alpha and interleukin-6 in women with the syndrome of hemolysis, elevated liver enzymes, and low platelet count. Acta Obstet Gynecol Scand. 1996; 75:695-701.
25. Guo R F, Ward P A. Role of C5a in inflammatory responses. Annu Rev Immunol. 2005; 23:821-852.
26. Oikonomopoulou K, Ricklin D, Ward P A, Lambris J D. Interactions between coagulation and complement—their role in inflammation. Semin Immunopathol. 2012; 34:151-165.
27. Rampersad R, Barton A, Sadovsky Y, Nelson D M. The C5b-9 membrane attack complex of complement activation localizes to villous trophoblast injury in vivo and modulates human trophoblast function in vitro. Placenta. 2008; 29:855-861.
28. Martel C, Cointe S, Maurice P, Matar S, Ghitescu M, Theroux P, Bonnefoy A. Requirements for membrane attack complex formation and anaphylatoxins binding to collagen-activated platelets. PLoS One. 6: e18812.
29. Peerschke E I, Yin W, Ghebrehiwet B. Complement activation on platelets: Implications for vascular inflammation and thrombosis. Molecular Immunol. 2010; 47:2170-2175.
30. Sims P J, Faioni E M, Wiedmer T, Shattil S J. Complement proteins C5b-9 cause release of membrane vesicles from the platelet surface that are enriched in the membrane receptor for coagulation factor Va and express prothrombinase activity. J Biol Chem. 1988; 263:18205-18212.
31. Nauta A J, Daha M R, Tijsma 0, van de Water B, Tedesco F, Roos A. The membrane attack complex of complement induces caspase activation and apoptosis. Eur J Immunol. 2002; 32:783-792.
32. Girardi G, Yarilin D, Thurman J M, Holers V M, Salmon J E. Complement activation induces dysregulation of angiogenic factors and causes fetal rejection and growth restriction. J Exp Med 2006; 203:2165-2175.
33. Maynard S E, Min J Y, Merchan J, Lim K H, Li J, Mondal S, Libermann T A, Morgan J P, Sellke F W, Stillman I E, Epstein F H, Sukhatme V P, Karumanchi S A. Excess placental soluble fms-like tyrosine kinase 1 (sFLT1) may contribute to endothelial dysfunction, hypertension, and proteinuria in preeclampsia. J Clin Invest. 2003; 111:649-658.
34. Levine R J, Maynard S E, Qian C, Lim K H, England L J, Yu K F, Schisterman E F, Thadhani R, Sachs B P, Epstein F H, Sibai B M, Sukhatme V P, Karumanchi S A. Circulating angiogenic factors and the risk of preeclampsia. N Engl J Med. 2004; 350:672-683.
35. Oppermann M, Gotze 0. Plasma clearance of the human C5a anaphylatoxin by binding to leucocyte C5a receptors. Immunology. 1994; 82:516-521.
36. Caritis S, Sibai B, Hauth J, Lindheimer M, VanDorsten P, Klebanoff M, Thom E, Landon M, Paul R, Miodovnik M, Meis P, Thurau G, Dombrowski M, McNellis D, Roberts J. Predictors of preeclampsia in women at high risk. National Institute of Child Health and Human Development Network of Maternal-Fetal Medicine Units. Am J Obstet Gynecol. 1998; 179:946-951.
37. Amara U, Flierl M A, Rittirsch D, Klos A, Chen H, Acker B, Bruckner U B, Nilsson B, Gebhard F, Lambris J D, Huber-Lang M. Molecular intercommunication between the complement and coagulation systems. J Immunol. 2010; 185:5628-5636.
38. Huber-Lang M, Sarma J V, Zetoune F S, Rittirsch D, Neff T A, McGuire S R, Lambris J D, Warner R L, Flierl M A, Hoesel L M, Gebhard F, Younger J G, Drouin S M, Wetsel R A, Ward P A. Generation of C5a in the absence of C3: a new complement activation pathway. Nat Med. 2006; 12:682-687.
39. Salmon J E, Heuser C, Triebwasser M, Liszewski M K, Kavanagh D, Roumenina L, Branch D W, Goodship T, Fremeaux-Bacchi V, Atkinson J P. Mutations in complement regulatory proteins predispose to preeclampsia: a genetic analysis of the PROMISSE cohort. PLoS Med. 2011; 8:e1001013.
40. vanWerkhoven M B, Damman J, Daha M R, Krikke C, van Goor H, van Son W J, Hillebrands J L, van Dijk M C, Seelen M A. Novel insights in localization and expression of C5aR and C5L2 under native and post-transplant conditions. Mol Immunol. 2013; 53:237-245.
41. Fayyazi A, Scheel O, Werfel T, Schweyer S, Oppermann M, Gotze O, Radzun H J, Zwirner J. The C5a receptor is expressed in normal renal proximal tubular but not in normal pulmonary or hepatic epithelial cells. Immunology. 2000; 99:38-45.
42. Li R, Coulthard L G, Wu M C, Taylor S M, Woodruff T M. C5L2: a controversial receptor of complement anaphylatoxin, C5a. FASEB J. 2013; 27:855-864.
43. Kerjaschki D, Schulze M, Binder S, Kain R, Ojha P P, Susani M, Horvat R, Baker P J, Couser W G. Transcellular transport and membrane insertion of the C5b-9 membrane attack complex of complement by glomerular epithelial cells in experimental membranous nephropathy. J Immunol. 1989; 143:546-552.
44. Morita Y, Ikeguchi H, Nakamura J, Hotta N, Yuzawa Y, Matsuo S. Complement activation products in the urine from proteinuric patients. J Am Soc Nephrol. 2000; 11:700-707.
45. Aggarwal P K, Jain V, Sakhuja V, Karumanchi S A, Jha V. Low urinary placental growth factor is a marker of pre-eclampsia. Kidney Int. 2006; 69:621-624.
46. Redman C W, Sacks G P, Sargent I L. Preeclampsia: an excessive maternal inflammatory response to pregnancy. Am J Obstet Gynecol. 1999; 180:499-506.
47. Marder S R, Chenoweth D E, Goldstein I M, Perez H D. Chemotactic responses of human peripheral blood monocytes to the complement-derived peptides C5a and C5a desArg. J Immunol. 1985; 134:3325-3326.
48. Sakuma M, Morooka T, Wang Y, Shi C, Croce K, Gao H, Strainic M, Medof M E, Simon D I. The intrinsic complement regulator decay-accelerating factor modulates the biological response to vascular injury. Arterioscler Thromb Vasc Biol. 2010; 30:1196-1202.
49. Kerr H, Richards A. Complement-mediated injury and protection of endothelium: Lessons from atypical haemolytic uraemic syndrome. Immunobiology. 2012; 217:195-203.
50. Sacks T, Moldow C F, Craddock P R, Bowers T K, Jacob H S. Oxygen radicals mediate endothelial cell damage by complement-stimulated granulocytes. An in vitro model of immune vascular damage. J Clin Invest. 1978; 61:1161-1167.
51. Gupta S, Aziz N, Sekhon L, Agarwal R, Mansour G, Li J, Agarwal A. Lipid peroxidation and antioxidant status in preeclampsia: a systematic review. Obstet Gynecol Surv. 2009; 64:750-759.
52. Burwick R M, Feinberg B B. Eculizumab for the treatment of severe preeclampsia/HELLP syndrome. Placenta. 2013; 34:201-203.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

The term “comprising” or “comprises” is used in reference to methods, and respective component(s) thereof, that are essential to the invention, yet open to the inclusion of unspecified elements, whether essential or not. The use of “comprising” indicates inclusion rather than limitation.

The term “consisting of” refers to methods, and respective components thereof as described herein, which are exclusive of any element not recited in that description of the embodiment.

1. A method of diagnosing preeclampsia or HELLP syndrome and optionally administering a preeclampsia therapy or HELLP syndrome therapy, comprising:
assaying a biological sample obtained from a subject to determine a level of a preeclampsia biomarker selected from the group consisting of kidney injury molecule 1 (KIM-1), a complement protein or component, and combinations thereof, wherein determining the level of the preeclampsia biomarker is by application of a reagent comprising a detectably labeled probe that reacts with the preeclampsia biomarker or a complex comprising the preeclampsia biomarker;
comparing the preeclampsia biomarker level to a reference level; and
diagnosing preeclampsia if the level of the preeclampsia biomarker is higher than the reference level.

2.-51. (canceled)

52. The method of claim 1, wherein the reagent is an antibody.

53. The method of claim 1, further comprising administering the preeclampsia therapy or HELLP syndrome therapy to treat preeclampsia or HELLP syndrome if preeclampsia or HELLP syndrome is diagnosed.

54. The method of claim 1, wherein the complement protein or component is C3a, C5a, C5b-9 or combinations thereof.

55. The method of claim 1, wherein the complement protein or component is a complement protein or component upstream from KIM-1.

56. The method of claim 1, wherein the complement protein or component is a complement protein or component downstream from KIM-1.

57. The method of claim 1, wherein the biological sample is urine and the level of the preeclampsia biomarker is normalized to urine creatinine level.

58. The method of claim 1, wherein the preeclampsia therapy or HELLP syndrome therapy comprises a complement protein inhibitor.

59. The method of claim 1, wherein the preeclampsia therapy or HELLP syndrome therapy comprises Eculizumab.

60. The method of claim 1, wherein the preeclampsia therapy or HELLP syndrome therapy comprises Pexelizumab.

61. The method of claim 1, wherein the preeclampsia therapy or HELLP syndrome therapy comprises Compstatin.

62. The method of claim 1, wherein the preeclampsia therapy or HELLP syndrome therapy comprises sCR1.

63. The method of claim 1, wherein the preeclampsia therapy or HELLP syndrome therapy comprises Heparin.

64. The method of claim 1, wherein the preeclampsia therapy or HELLP syndrome therapy comprises magnesium sulfate, an antihypertensive drug, or combinations thereof.

65. The method of claim 1, wherein the preeclampsia therapy or HELLP syndrome therapy comprises hydralazine and labetalol, nifedipine, sodium nitroprusside or a combination thereof.

66. The method of claim 1, further comprising:
assaying the biological sample to determine the level of an additional preeclampsia biomarker selected from the group consisting of CD46, CD55, and CD59, PlGF, PP13, PAPP-A, Inhibin A, Activin A, MMP-9, Endothelin-1, P-selectin, NGAL, sEng, triglycerides, HDL cholesterol, LDL cholesterol, hPL, SP-1, hCG, sL-selectin, ApoB, TGF-β1, TNF-R1, TNF-R2, complement activation product Bb, triglycerides, PAI-2, IGFBP-1, free foetal DNA, sFlt-1, sEng, BNP, and combinations thereof; and
comparing the level of the additional preeclampsia biomarker to a reference level for the additional preeclampsia biomarker; and
wherein diagnosing preeclampsia or HELLP syndrome comprises diagnosing preeclampsia or HELLP syndrome if the level of the preeclampsia biomarker is higher than the reference level and if the level of the additional preeclampsia biomarker differs from the reference level for the additional preeclampsia biomarker.

67. The method of claim 1, further comprising:
assaying the biological sample to determine the level of an additional preeclampsia biomarker selected from the group consisting of PlGF, PP13, PAPP-A, Inhibin A, Activin A, MMP-9, Endothelin-1, P-selectin, NGAL, sEng, triglycerides, HDL cholesterol, LDL cholesterol, and combinations thereof.

68. The method of claim 1, further comprising:
diagnosing preeclampsia or HELLP syndrome if the level of the preeclampsia biomarker is higher than the reference level, and
if the preeclampsia biomarker is selected from the group consisting of PlGF, PP13, PAPP-A, HDL cholesterol, and combinations thereof, and the level of the preeclampsia biomarker is lower than the reference level, or
if the preeclampsia biomarker is selected from the group consisting of Inhibin A, Activin A, MMP-9, Endothelin-1, P-selectin, sEng, NGAL, triglycerides, LDL cholesterol and combinations thereof, and the level of the preeclampsia biomarker is higher than the reference level.

69. A kit for diagnosing preeclampsia or HELLP syndrome in a subject in need thereof, comprising: one or more reagents comprising at least one detectably labeled probe for the detection of KIM-1, one or more complement proteins or components, or a combination thereof.

70. An assay for diagnosing preeclampsia or HELLP syndrome, comprising:
a solid phase;
a first reagent to react with a preeclampsia biomarker, wherein the first reagent is immobilized on the solid phase;
a second reagent to specifically react with a preeclampsia biomarker, wherein the second reagent comprise a label;
a substrate to react with the label; and
a third reagent to react with the second reagent to serve as a control.
